SEQUENCE LISTING (15 sequences)

SEQ ID NO: 1 - protein, 76 aa, Homo sapiens [sequence omitted]

SEQ ID NO: 2 - protein, 152 aa, Homo sapiens
  UNSURE (1)..(152)
MAGLPRRIIKETQRLLAEPVPGIKAEPDESNARYFHVVIAGPQDSPFEGGTFKLELFLPEEYPMAAPKVRFMTKIYHPNVDKLGRICLDILKDKWSPALQIRTVLLSIQALLSAPNPDDPLANDVAEQWKTNEAQAIETARAWTRLYAMNNI

SEQ ID NO: 3 - protein, 170 aa, Homo sapiens
  unsure (1)..(170)
MPGEVQASYLKSQSKLSDEGRLEPRKFHCKGVKVPRNFRLLEELEEGQKGVGDGTVSWGLEDDEDMTLTRWTGMIIGPPRTIYENRIYSLKIECGPKYPEAPPFVRFVTKINMNGVNSSNGVVDPRAISVLAKWQNSYSIKVVLQELRRLMMSKENMKLPQPPEGQCYSN

SEQ ID NO: 4 - protein, 1058 aa, Homo sapiens
  unsure (1)..(1058)
MSSSPLSKKRRVSGPDPKPGSNCSPAQSVLSEVPSVPTNGMAKNGSEADIDEGLYSRQLYVLGHEAMKRLQTSSVLVSGLRGLGVEIAKNIILGGVKAVTLHDQGTAQWADLSSQFYLREEDIGKNRAEVSQPRLAELNSYVPVTAYTGPLVEDFLSGFQVVVLTNTPLEDQLRVGEFCHNRGIKLVVADTRGLFGQLFCDFGEEMILTDSNGEQPLSAMVSMVTKDNPGVVTCLDEARHGFESGDFVSFSEVQGMVELNGNQPMEIKVLGPYTFSICDTSNFSDYIRGGIVSQVKVPKKISFKSLVASLAEPDFVVTDFAKFSRPAQLHIGFQALHQFCAQHGRPPRPRNEEDAAELVALAQAVNARALPAVQQNNLDEDLIRKLAYVAAGDLAPINAFIGGLAAQEVMKACSGKFMPIMQWLYFDALECLPEDKEVLTEDKCLQRQNRYDGQVAVFGSDLQEKLGKQKYFLVGAGAIGCELLKNFAMIGLGCGEGGEIIVTDMDTIEKSNLNRQFLFRPWDVTKLKSDTAAAAVRQMNPHIRVTSHQNRVGPDTERIYDDDFFQNLDGVANALDNVDARMYMDRRCVYYRKPLLESGTLGTKGNVQVVIPFLTESYSSSQDPPEKSIPICTLKNFPNAIEHTLQWARDEFEGLFKQPAENVNQYLTDPKFVERTLRLAGTQPLEVLEAVQRSLVLQRPQTWADCVTWACHHWHTQYSNNIRQLLHNFPPDQLTSSGAPFWSGPKRCPHPLTFDVNNPLHLDYVMAAANLFAQTYGLTGSQDRAAVATFLQSVQVPEFTPKSGVKIHVSDQELQSANASVDDSRLEELKATLPSPDKLPGFKMYPIDFEKDDDSNFHMDFIVAASNLRAENYDIPSADRHKSKLIAGKIIPAIATTTAAVVGLVCLELYKVVQGHRQLDSYKNGFLNLALPFFGFSEPLAAPRHQYYNQEWTLWDRFEVQGLQPNGEEMTLKQFLDYFKTEHKLEITMLSQGVSMLYSFFMPAAKLKERLDQPMTEIVSRVSKRKLGRHVRALVLELCCNDESGEDVEVPYVRYTIR

SEQ ID NO: 5 - protein, 640 aa, Homo sapiens
  unsure (1)..(640)
MALSRGLPRELAEAVAGGRVLVVGAGGIGCELLKNLVLTGFSHIDLIDLDTIDVSNLNRQFLFQKKHVGRSKAQVAKESVLQFYPKANIVAYHDSIMNPDYNVEFFRQFILVMNALDNRAARNHVNRMCLAADVPLIESGTAGYLGQVTTIKKGVTECYECHPKPTQRTFPGCTIRNTPSEPIHCIVWAKYLFNQLFGEEDADQEVSPDRADPEAAWEPTEAEARARASNEDGDIKRISTKEWAKSTGYDPVKLFTKLFKDDIRYLLTMDKLWRKRKPPVPLDWAEVQSQGEETNASDQQNEPQLGLKDQQVLDVKSYARLFSKSIETLRVHLAEKGDGAELIWDKDDPSAMDFVTSAANLRMHIFSMNMKSRFDIKSMAGNIIPAIATTNAVIAGLIVLEGLKILSGKIDQCRTIFLNKQPNPRKKLLVPCALDPPNPNCYVCASKPEVTVRLNVHKVTVLTLQDKIVKEKFAMVAPDVQIEDGKGTILISSEEGETEANNHKKLSEFGIRNGSRLQADDFLQDYTLLINILHSEDLGKDVEFEVVGDAPEKVGPKQAEDAAKSITNGSDDGAQPSTSTAQEQDDVLIVDSDEEDSSNNADVSEEERSRKRKLDEKENLSAKRSRIEQKEELDDVIALD

SEQ ID NO: 6 - protein, 103 aa, Homo sapiens
  unsure (1)..(103)
MKEDLNLENFTAKTIYENRIYSLKIECGPKYPEAPPFVRFVTKINMNGVNSSNGVVDPRAISVLAKWQNSYSIKVVLQELRRLMMSKENMKLPQPPEGQCYSN

SEQ ID NO: 7 - protein, 147 aa, Homo sapiens
  unsure (1)..(147)
MAATTGSGVKVPRNFRLLEELEEGQKGVGDGTVSWGLEDDEDMTLTRWTGMIIGPPRTIYENRIYSLKIECGPKYPEAPPFVRFVTKINMNGVNSSNGVVDPRAISVLAKWQNSYSIKVVLQELRRLMMSKENMKLPQPPEGQCYSN

SEQ ID NO: 8 - DNA, 2539 bp, Homo sapiens
  Unsure (1)..(2539): Human Uevla variant 1
cccgcctaacctcttcctgcgatgagctcggcacgggaattattattgtcaattttacttgcaagaagtttcctacaagagccaaggaatccatgcgagtaaacatttacgggcaccatagataaaaggcttgtgttttaatcctcatcctctccacctgttagctctgagtctcagttttctcatctctaaaaatggggatattcacaggagttgctgcatcgagttgtgaggattaaaagttggatgtaacggcttggtaattatgagctcttctagtgtcccttcctcttccctgtgcccaaggggttttaggaaagcattttatctccacagcaatcctatgaggttgatactactatcctcatagaaggggaaactgatgccaggagaggttcaagcgtcttacctgaagtcacaaagcaaactgagtgatgaaggaagacttgaacctagaaaatttcactgcaaaggagtaaaagtccctcgcaatttccgactgttggaagaactcgaagaaggccagaaaggagtaggagatggcacagttagctggggtctagaagatgacgaagacatgacacttacaagatggacagggatgataattgggcctccaagaacaatttatgaaaaccgaatatacagccttaaaatagaatgtggacctaaatacccagaagcacccccctttgtaagatttgtaacaaaaattaatatgaatggagtaaatagttctaatggagtggtggacccaagagccatatcagtgctagcaaaatggcagaattcatatagcatcaaagttgtcctgcaagagcttcggcgcctaatgatgtctaaagaaaatatgaaactccctcagccgcccgaaggacagtgttacagcaattaatcaaaaagaaaaaccacaggcccttccccttccccccaattcgatttaatcagtcttcattttccacagtagtaaattttctagatacgtcttgtagacctcaaagtaccggaaaggaagctcccattcaaaggaaatttatcttaagatactgtaaatgatactaattttttgtccatttgaaatatataagttgtgctataacaaatcatcctgtcaagtgtaaccactgtccacgtagttgaacttctgggatcaagaaagtctatttaaattgattcccatcataactggtggggcacatctaactcaactgtgaaaagacacatcacacaatcaccttgctgctgattacacggcctggggtctctgccttctccccttaccctcccgcctcccaccctccctgcaacaacagccctctagcctggggggcttgttagagtagatgtgaaggtttcaggtcgcagcctgtgggactactgctaggtgtgtggggtgtttcgcctgcacccctggtttctttaagtcttaagtgatgccccttccaaaccatcatcctgtccccacgctcctccactcccgcccttggccgaagcatagattgtaacccctccactcccctctgagattggccttcggtgaggaattcagggctttccccatatcttctctcccccacctttatcgaggggtgctgctttttctccctcctcctcaagttcctttttgcaccgtcaccacccaacaccttccatgacacttccttgctttggccagaagccatcaggtaaggttggaaagagcctctgacctcccttgtttagttttggaaccatactcactcactctccaccagcctgggaaatgaatattgggtcctcagccctgccaccctctgctgtcatcagctgatgcattgtttttagctcaggttttgataaggtgaaaagaatagtcaccagggttactcagacctgccagctctcggagtccttggtggttgaacttggagaaagaccgcatgaagatacttgtaagcacacatgatccctctgaattgttttactttcctgtaactgcttttgcttttaaaaattgaagaagttttaaacagggctttcatttggtcatccttgcaatccattggggtctagtttggaatctgacaactggaacaaaaagaaccttgaatccggtgcatgccttggttttggtgctgctgctgcttcccaagatcctcagcagggattaagaaggaacccggtgtgcacagcagatccccgaaattggtgggcttgacctcctggcaaattgctgcgtctttccacttgctgttcaggaccactaaatgctgaaatgtggatgcataccgaaataaaagcaattcattgtgtactaaaggttttttttttttttttaatttagtatttgtgtaaaaccaccttttgaagcagcaactatcaagtctgaaaagcaattgatgtttccattaatctttttctggggggaaaaccttagttctaaggatttaacatcctgtaagtgaagtttaacataacagtattccataagcagcctttttattgtcagaccattgcctgattttaatataataaaaaaaaagtgtgcgttaatatttaa

SEQ ID NO: 9 - DNA, 2477 bp, Homo sapiens
  unsure (1)..(2477): Human Uevla variant 2
cccgcctaacctcttcctgcgatgagctcggcacgggaattattattgtcaattttacttgcaagaagtttcctacaagagccaaggaatccatgcgagtaaacatttacgggcaccatagataaaaggcttgtgttttaatcctcatcctctccacctgttagctctgagtctcagttttctcatctctaaaaatggggatattcacaggagttgctgcatcgagttgtgaggattaaaagttggatgtaacggcttgcaatcctatgaggttgatactactatcctcatagaaggggaaactgatgccaggagaggttcaagcgtcttacctgaagtcacaaagcaaactgagtgatgaaggaagacttgaacctagaaaatttcactgcaaaggagtaaaagtccctcgcaatttccgactgttggaagaactcgaagaaggccagaaaggagtaggagatggcacagttagctggggtctagaagatgacgaagacatgacacttacaagatggacagggatgataattgggcctccaagaacaatttatgaaaaccgaatatacagccttaaaatagaatgtggacctaaatacccagaagcacccccctttgtaagatttgtaacaaaaattaatatgaatggagtaaatagttctaatggagtggtggacccaagagccatatcagtgctagcaaaatggcagaattcatatagcatcaaagttgtcctgcaagagcttcggcgcctaatgatgtctaaagaaaatatgaaactccctcagccgcccgaaggacagtgttacagcaattaatcaaaaagaaaaaccacaggcccttccccttccccccaattcgatttaatcagtcttcattttccacagtagtaaattttctagatacgtcttgtagacctcaaagtaccggaaaggaagctcccattcaaaggaaatttatcttaagatactgtaaatgatactaattttttgtccatttgaaatatataagttgtgctataacaaatcatcctgtcaagtgtaaccactgtccacgtagttgaacttctgggatcaagaaagtctatttaaattgattcccatcataactggtggggcacatctaactcaactgtgaaaagacacatcacacaatcaccttgctgctgattacacggcctggggtctctgccttctccccttaccctcccgcctcccaccctccctgcaacaacagccctctagcctggggggcttgttagagtagatgtgaaggtttcaggtcgcagcctgtgggactactgctaggtgtgtggggtgtttcgcctgcacccctggtttctttaagtcttaagtgatgccccttccaaaccatcatcctgtccccacgctcctccactcccgcccttggccgaagcatagattgtaacccctccactcccctctgagattggccttcggtgaggaattcagggctttccccatatcttctctcccccacctttatcgaggggtgctgctttttctccctcctcctcaagttcctttttgcaccgtcaccacccaacaccttccatgacacttccttgctttggccagaagccatcaggtaaggttggaaagagcctctgacctcccttgtttagttttggaaccatactcactcactctccaccagcctgggaaatgaatattgggtcctcagccctgccaccctctgctgtcatcagctgatgcattgtttttagctcaggttttgataaggtgaaaagaatagtcaccagggttactcagacctgccagctctcggagtccttggtggttgaacttggagaaagaccgcatgaagatacttgtaagcacacatgatccctctgaattgttttactttcctgtaactgcttttgcttttaaaaattgaagaagttttaaacagggctttcatttggtcatccttgcaatccattggggtctagtttggaatctgacaactggaacaaaaagaaccttgaatccggtgcatgccttggttttggtgctgctgctgcttcccaagatcctcagcagggattaagaaggaacccggtgtgcacagcagatccccgaaattggtgggcttgacctcctggcaaattgctgcgtctttccacttgctgttcaggaccactaaatgctgaaatgtggatgcataccgaaataaaagcaattcattgtgtactaaaggttttttttttttttttaatttagtatttgtgtaaaaccaccttttgaagcagcaactatcaagtctgaaaagcaattgatgtttccattaatctttttctggggggaaaaccttagttctaaggatttaacatcctgtaagtgaagtttaacataacagtattccataagcagcctttttattgtcagaccattgcctgattttaatataataaaaaaaaagtgtgcgttaatatttaaaaaaaaaaaaaaaaa

SEQ ID NO: 10 - DNA, 2179 bp, Homo sapiens
  unsure (1)..(2179): Human Uev1b
gcctaacctcttcctgcgatgagctcggcacgggaattattattgtcaattttacttgcaagaagtttcctacaagagccaaggaatccatgcgagtaaacatttacgggcaccatagataaaagcaatcctatgaggttgatactactatcctcatagaaggggaaactgatgccaggagaggttcaagcgtcttacctgaagtcacaaagcaaactgagtgatgaaggaagacttgaacctagaaaatttcactgcaaagacaatttatgaaaaccgaatatacagccttaaaatagaatgtggacctaaatacccagaagcacccccctttgtaagatttgtaacaaaaattaatatgaatggagtaaatagttctaatggagtggtggacccaagagccatatcagtgctagcaaaatggcagaattcatatagcatcaaagttgtcctgcaagagcttcggcgcctaatgatgtctaaagaaaatatgaaactccctcagccgcccgaaggacagtgttacagcaattaatcaaaaagaaaaaccacaggcccttccccttccccccaattcgatttaatcagtcttcattttccacagtagtaaattttctagatacgtcttgtagacctcaaagtaccggaaaggaagctcccattcaaaggaaatttatcttaagatactgtaaatgatactaattttttgtccatttgaaatatataagttgtgctataacaaatcatcctgtcaagtgtaaccactgtccacgtagttgaacttctgggatcaagaaagtctatttaaattgattcccatcataactggtggggcacatctaactcaactgtgaaaagacacatcacacaatcaccttgctgctgattacacggcctggggtctctgccttctccccttaccctcccgcctcccaccctccctgcaacaacagccctctagcctggggggcttgttagagtagatgtgaaggtttcaggtcgcagcctgtgggactactgctaggtgtgtggggtgtttcgcctgcacccctggtttctttaagtcttaagtgatgccccttccaaaccatcatcctgtccccacgctcctccactcccgcccttggccgaagcatagattgtaacccctccactcccctctgagattggccttcggtgaggaattcagggctttccccatatcttctctcccccacctttatcgaggggtgctgctttttctccctcctcctcaagttcctttttgcaccgtcaccacccaacaccttccatgacacttccttgctttggccagaagccatcaggtaaggttggaaagagcctctgacctcccttgtttagttttggaaccatactcactcactctccaccagcctgggaaatgaatattgggtcctcagccctgccaccctctgctgtcatcagctgatgcattgtttttagctcaggttttgataaggtgaaaagaatagtcaccagggttactcagacctgccagctctcggagtccttggtggttgaacttggagaaagaccgcatgaagatacttgtaagcacacatgatccctctgaattgttttactttcctgtaactgcttttgcttttaaaaattgaagaagttttaaacagggctttcatttggtcatccttgcaatccattggggtctagtttggaatctgacaactggaacaaaaagaaccttgaatccggtgcatgccttggttttggtgctgctgctgcttcccaagatcctcagcagggattaagaaggaacccggtgtgcacagcagatccccgaaattggtgggcttgacctcctggcaaattgctgcgtctttccacttgctgttcaggaccactaaatgctgaaatgtggatgcataccgaaataaaagcaattcattgtgtactaaaggttttttttttttttttaatttagtatttgtgtaaaaccaccttttgaagcagcaactatcaagtctgaaaagcaattgatgtttccattaatctttttctggggggaaaaccttagttctaaggatttaacatcctgtaagtgaagtttaacataacagtattccataagcagcctttttattgtcagaccattgcctgattttaatataataaaaaaaaagtgtgcgttaatatttaa

SEQ ID NO: 11 - DNA, 2138 bp, Homo sapiens
  unsure (1)..(2138): Human Uev1d
gggggggtgaagaaggggccggccttcaagcaagagcgacgcaagatggcagccaccacgggctcgggagtaaaagtccctcgcaatttccgactgttggaagaactcgaagaaggccagaaaggagtaggagatggcacagttagctggggtctagaagatgacgaagacatgacacttacaagatggacagggatgataattgggcctccaagaacaatttatgaaaaccgaatatacagccttaaaatagaatgtggacctaaatacccagaagcacccccctttgtaagatttgtaacaaaaattaatatgaatggagtaaatagttctaatggagtggtggacccaagagccatatcagtgctagcaaaatggcagaattcatatagcatcaaagttgtcctgcaagagcttcggcgcctaatgatgtctaaagaaaatatgaaactccctcagccgcccgaaggacagtgttacagcaattaatcaaaaagaaaaaccacaggcccttccccttccccccaattcgatttaatcagtcttcattttccacagtagtaaattttctagatacgtcttgtagacctcaaagtaccggaaaggaagctcccattcaaaggaaatttatcttaagatactgtaaatgatactaattttttgtccatttgaaatatataagttgtgctataacaaatcatcctgtcaagtgtaaccactgtccacgtagttgaacttctgggatcaagaaagtctatttaaattgattcccatcataactggtggggcacatctaactcaactgtgaaaagacacatcacacaatcaccttgctgctgattacacggcctggggtctctgccttctccccttaccctcccgcctcccaccctccctgcaacaacagccctctagcctggggggcttgttagagtagatgtgaaggtttcaggtcgcagcctgtgggactactgctaggtgtgtggggtgtttcgcctgcacccctggtttctttaagtcttaagtgatgccccttccaaaccatcatcctgtccccacgctcctccactcccgcccttggccgaagcatagattgtaacccctccactcccctctgagattggccttcggtgaggaattcagggctttccccatatcttctctcccccacctttatcgaggggtgctgctttttctccctcctcctcaagttcctttttgcaccgtcaccacccaacaccttccatgacacttccttgctttggccagaagccatcaggtaaggttggaaagagcctctgacctcccttgtttagttttggaaccatactcactcactctccaccagcctgggaaatgaatattgggtcctcagccctgccaccctctgctgtcatcagctgatgcattgtttttagctcaggttttgataaggtgaaaagaatagtcaccagggttactcagacctgccagctctcggagtccttggtggttgaacttggagaaagaccgcatgaagatacttgtaagcacacatgatccctctgaattgttttactttcctgtaactgcttttgcttttaaaaattgaagaagttttaaacagggctttcatttggtcatccttgcaatccattggggtctagtttggaatctgacaactggaacaaaaagaaccttgaatccggtgcatgccttggttttggtgctgctgctgcttcccaagatcctcagcagggattaagaaggaacccggtgtgcacagcagatccccgaaattggtgggcttgacctcctggcaaattgctgcgtctttccacttgctgttcaggaccactaaatgctgaaatgtggatgcataccgaaataaaagcaattcattgtgtactaaaggttttttttttttttttaatttagtatttgtgtaaaaccaccttttgaagcagcaactatcaagtctgaaaagcaattgatgtttccattaatctttttctggggggaaaaccttagttctaaggatttaacatcctgtaagtgaagtttaacataacagtattccataagcagcctttttattgtcagaccattgcctgattttaatataataaaaaaaaagtgtgcgttaatatttaaaaaaa

SEQ ID NO: 12 - DNA, 3544 bp, Homo sapiens
  unsure (1)..(3544): Human E1 (UBA1)
tcccagacccggggctctccaaggccccgcgcttccgagctccgcgcaaactctggcttctcttgtacgacagaggtggtttgctcttccgttgccccgtggcttcagctcatctttggcaggaaggcgaggcttccgcccggcacaggggatgtccagctcgccgctgtccaagaaacgtcgcgtgtccgggcctgatccaaagccgggttctaactgctcccctgcccagtccgtgttgtccgaagtgccctcggtgccaaccaacggaatggccaagaacggcagtgaagcagacatagacgagggcctttactcccggcagctgtatgtgttgggccatgaggcaatgaagcggctccagacatccagtgtcctggtatcaggcctgcggggcctgggcgtggagatcgctaagaacatcatccttggtggggtcaaggctgttaccctacatgaccagggcactgcccagtgggctgatctttcctcccagttctacctgcgggaggaggacatcggtaaaaaccgggccgaggtatcacagccccgcctcgctgagctcaacagctatgtgcctgtcactgcctacactggacccctcgttgaggacttccttagtggtttccaggtggtggtgctcaccaacacccccctggaggaccagctgcgagtgggtgagttctgtcacaaccgtggcatcaagctggtggtggcagacacgcggggcctgtttgggcagctcttctgtgactttggagaggaaatgatcctcacagattccaatggggagcagccactcagtgctatggtttctatggttaccaaggacaaccccggtgtggttacctgcctggatgaggcccgacacgggtttgagagcggggactttgtctccttttcagaagtacagggcatggttgaactcaacggaaatcagcccatggagatcaaagtcctgggtccttatacctttagcatctgtgacacctccaacttctccgactacatccgtggaggcatcgtcagtcaggtcaaagtacctaagaagattagctttaaatccttggtggcctcactggcagaacctgactttgtggtgacggacttcgccaagttttctcgccctgcccagctgcacattggcttccaggccctgcaccagttctgtgctcagcatggccggccacctcggccccgcaatgaggaggatgcagcagaactggtagccttagcacaggctgtgaatgctcgagccctgccagcagtgcagcaaaataacctggacgaggacctcatccggaagctggcatatgtggctgctggggatctggcacccataaacgccttcattgggggcctggctgcccaggaagtcatgaaggcctgctccgggaagttcatgcccatcatgcagtggctatactttgatgcccttgagtgtctccctgaggacaaagaggtcctcacagaggacaagtgcctccagcgccagaaccgttatgacgggcaagtggctgtgtttggctcagacctgcaagagaagctgggcaagcagaagtatttcctggtgggtgcgggggccattggctgtgagctgctcaagaactttgccatgattgggctgggctgcggggagggtggagaaatcatcgttacagacatggacaccattgagaagtcaaatctgaatcgacagtttcttttccggccctgggatgtcacgaagttaaagtctgacacggctgctgcagctgtgcgccaaatgaatccacatatccgggtgacaagccaccagaaccgtgtgggtcctgacacggagcgcatctatgatgacgattttttccaaaacctagatggcgtggccaatgccctggacaacgtggatgcccgcatgtacatggaccgccgctgtgtctactaccggaagccactgctggagtcaggcacactgggcaccaaaggcaatgtgcaggtggtgatccccttcctgacagagtcgtacagttccagccaggacccacctgagaagtccatccccatctgtaccctgaagaacttccctaatgccatcgagcacaccctgcagtgggctcgggatgagtttgaaggcctcttcaagcagccagcagaaaatgtcaaccagtacctcacagaccccaagtttgtggagcgaacactgcggctggcaggcactcagcccttggaggtgctggaggctgtgcagcgcagcctggtgctgcagcgaccacagacctgggctgactgcgtgacctgggcctgccaccactggcacacccagtactcgaacaacatccggcagctgctgcacaacttccctcctgaccagctcacaagctcaggagcgccgttctggtctgggcccaaacgctgtccacacccgctcacctttgatgtcaacaatcccctgcatctggactatgtgatggctgctgccaacctgtttgcccagacctacgggctgacaggctctcaggaccgagctgctgtggccacattcctgcagtctgtgcaggtccccgaattcacccccaagtctggcgtcaagatccatgtttctgaccaggagctgcagagcgccaatgcctctgttgatgacagtcgtctagaggagctcaaagccactctgcccagcccagacaagctccctggattcaagatgtaccccattgactttgagaaggatgatgacagcaactttcatatggatttcatcgtggctgcatccaacctccgggcagaaaactatgacattccttctgcagaccggcacaagagcaagctgattgcagggaagatcatcccagccattgccacgaccacagcagccgtggttggccttgtgtgtctggagctgtacaaggttgtgcaggggcaccgacagcttgactcctacaagaatggtttcctcaacttggccctgcctttctttggtttctctgaaccccttgccgcaccacgtcaccagtactataaccaagagtggacattgtgggatcgctttgaggtacaagggctgcagcctaatggtgaggagatgaccctcaaacagttcctcgactattttaagacagagcacaaattagagatcaccatgctgtcccagggcgtgtccatgctctattccttcttcatgccagctgccaagctcaaggaacggttggatcagccgatgacagagattgtgagccgtgtgtcgaagcgaaagctgggccgccacgtgcgggcgctggtgcttgagctgtgctgtaacgacgagagcggcgaggatgtcgaggttccctatgtccgatacaccatccgctgaccccgtctgctcctctaggctggccccttgtccacccctctccacaccccttccagcccagggttcccatttggcttctggcagtggcccaactagccaagtctggtgttccctcatcatccccctacctgaacccctcttgccactgccttctaccttgtttgaaacctgaatcctaataaagaattaataactcccaaaaaaaaaaaaaaaaaa

SEQ ID NO: 13 - DNA, 2558 bp, Homo sapiens
  unsure (1)..(2558): Human E2 (Ubc13)
cgcgcgcgcagtcgcgcgcgggtcgtgccgtaccaccgtcgcgggcaggctcggccacgagcgccagagccccgcgcctcccctcgcggcctgtcccaagtccctgccccgcaacagagcgtcacttccgccatccccggcagcggttggggcggggcgcacgggggagggggccaggtcggagggaagcccgcccgtgcccgagcccgcgcccgagcagggactacatttcccgaggggcctcggcggcggctgcggcgacgggcgcggcaacgtcccccggaagtggagcccgggacttccactcgtgcgtgaggcgagaggagccggagacgagaccagaggccgaactcgggttctgacaagatggccgggctgccccgcaggatcatcaaggaaacccagcgtttgctggcagaaccagttcctggcatcaaagccgaaccagatgagagcaacgcccgttattttcatgtggtcattgctggccctcaggattccccctttgagggagggacttttaaacttgaactattccttccagaagaatacccaatggcagcccctaaagtacgtttcatgaccaaaatttatcatcctaatgtagacaagttgggaagaatatgtttagatattttgaaagataagtggtccccagcactgcagatccgcacagttctgctatcgatccaggccttgttaagtgctcccaatccagatgatccattagcaaatgatgtagcggagcagtggaagaccaacgaagcccaagccatagaaacagctagagcatggactaggctatatgccatgaataatatttaaattgatacgatcatcaagtgtgcatcacttctcctgttctgccaagacttcctcctctttgtttgcatttaatggacacagtcttagaaacattacagaataaaaaagcccagacatcttcagtcctttggtgattaaatgcacattagcaaatctatgtcttgtcctgattcactgtcataaagcatgagcagaggctagaagtatcatctggattgttgtgaaacgtttaaaagcagtggcccttattcatttcccccatcctggtttaagtataaagcactgtgaatgaaggtagttgtcaggttagctgcaggggtgtgggtgtttttattttattttattttattttatttttgaggggggaggtagtttaattttatgggctcctttcccccttttttggtgatctaattgcattggttaaaagcagctaaccaggtctttagaatatgctctagccaagtctaactttatttagacgctgtagatggacaagcttgattgttggaaccaaaatgggaacattaaacaaacatcacagccctcactaataacattgctgtcaagtgtagattccccccttcaaaaaaagcttgtgaccattttgtatggcttgtctggaaacttctgtaaatcttatgttttagtaaaatattttttgttattctactttgcctttgtacagtttattttactgtgtttatttcattttcccaatttgacaatcgtattttaaaattgaaactgatggaacattctttcttggtcttcaccatctgacaaattgaatggcaagaggtggattttgccagtttcttttcactgatgcagatttgtgttaagatagtactgaatggagtatttataaactggccctgagcatgcataaagcatcagtatctgacctttttttaaccttctaggaatttgaaataaatgtgtttgtgttgtctgattagatgatcattggtgtcttgccacaatgtttaaaaattactgtacaggaaagtcacagcaaagatagcagttgtgactgacatgtaggactttcacagttgtgccacatttttgcctaaaatttgggttatgacatttttcttggttcttatctgaaaatttcatctgtaacctttcatgtgtgttaagaaacactgatctgatcatttgggatttgctgaggcatttgtgagtcttccttataaacctgatgagcagatctcaactatctagcttgtgtgtcatcagaaaggtttatccctttgagagtatcaagtcctcagttaatgattcttgctttcatccctccagtatttgctgtgggagctcgttttattctttaatttggaattcagtaatttttcttctttattgacgaattcctcccctcacaaaactgttctttcccacctctctccatatctaattcctgattcttgttatttttaagtcataaatgtagccagtcataaatacataaatgttaaccttcgggttgcaaccttgtctcttgcagtttaaggtaatggatattgtagcccatttgaattttcttcactcttattctcgtaattctggagtttcttcagattgtggtgtattttattgtgctcctatgtaagatgaagaattaactattaaaattacattttcaacatacaaaagcttttgatgactggtaactggtatccttccaaataaatgcattgcttggtaaaaaaaaaaaaaaaaaa

SEQ ID NO: 14 - DNA, 1535 bp, Homo sapiens
  unsure (1)..(1535): Human ubiquitin E2 variant 2 (Mms2)
cgcgtcgggctgcaggagaagatggcggtctccacaggagttaaagttcctcgtaattttcgcttgttggaagaacttgaagaaggacaaaaaggagtaggcgacggtacagttagctggggccttgaagatgatgaagatatgacacttacaaggtggacaggcatgattattgggccaccaaggacaaattatgaaaacagaatatatagcctgaaagtagaatgtggacctaaatacccagaagctcctccgtcagttagatttgtaacaaaaattaatatgaacggaataaataattccagtgggatggtggatgcccggagcataccagtgttagcaaaatggcaaaattcatatagcattaaagttgtacttcaagagctaagacgtctaatgatgtccaaagaaaatatgaagcttccacagccaccagaaggacaaacatacaacaattaattttagtggatctcaaacttgtcttaaatcaacaaccttctactcatgttaatgtcttgattaaatatcacaatgcaaaatacacattaagtaaaagaattccagctggtaaacatgacctggacatttgtaagaatatatttaatatatgtacacccattatgttttcaggtaacaggaggaaaaatgcagcacaattttttttctcttgaaaggcactgtcatttaaacataaacctggagtactcgaaatagaattcaggtttacaagatgaaagcgtgtggagaagtgtcagatggcagtggaagcatgtgtgtttctaaaaagtaaaaatctcaagaaaacagaaatggcatgctttacccatcttacttagtgaaagagagctgcagttgaaattgtttaaaaagtagcaggtacaatgaatattgtcacagatgtgttaatttttgaagcaatgtgggtgctgactactagtagtatcaaaaatatgttcaggattgttttgatacctgtatttataataaaaaatgttggggggagttgatgaattcctgttaaaagctgttcttgtgtgttacatgtaacagacatggtaaatatttgtttacagtctttgtttaacaaaccatgcatttaagtttaagtgaagtcaacaaaaaggaaataggtgtatggatatgtgattttgagattaaagttagtcttaaaatgtaaataaaatgtgaaacgtgtcctcagagactgtgccatttctattatgttgatgtatatgtacagtaccttgccagggaagcaaaaattggaattattgtagcttttcatgtatacacacttttatttaccctattttgtgtacttcttgtgaattataatttgcagactatttcagaaaagaaattatctagtttaatttcttctttggacaaggagtcctaggtattatattttgagtttgatttcaccagaaataataatattaaaaagatctttgcattctggcagttcttttaggattataggttgcaaattatccaaatatatatcccattttttaaagcataaaaaaaaaaaa

SEQ ID NO: 15 - protein, 145 aa, Homo sapiens
  unsure (1)..(145): Human ubiquitin E2 variant 2 (Mms)
MAVSTGVKVPRNFRLLEELEEGQKGVGDGTVSWGLEDDEDMTLTRWTGMIIGPPRTNYENRIYSLKVECGPKYPEAPPSVRFVTKINMNGINNSSGMVDARSIPVLAKWQNSYSIKVVLQELRRLMMSKENMKLPQPPEGQTYNN

(19) United States
(12) Patent Application Publication
(10) Pub. No.: US 2009/0269731 A1
(43) Pub. Date: Oct. 29, 2009

(54) E3-INDEPENDENT UBIQUITINYLATION ASSAY

(75) Inventor: John C. Reed, Rancho Santa Fe, CA (US)

Correspondence Address: PATENT CORRESPONDENCE;ARNALL GOLDEN GREGORY LLP, 171 17TH STREET NW, SUITE 2100, ATLANTA, GA, 30363, US

(73) Assignee: Burnham Institute for Medical Research, La Jolla, CA (US)

(21) Appl. No.: 12/432,493

(22) Filed: Apr. 29, 2009

Related U.S. Application Data
(60) Provisional application No. 61/048,796, filed on Apr. 29, 2008.

(51) Int. Cl. C12Q 1/00

(52) U.S. Cl. 435/4

(57) ABSTRACT

Disclosed herein are compositions and methods for assaying ubiquitination independent of ubiquitin ligase (E3) or a target protein.

### CROSS-REFERENCE TO RELATED APPLICATIONS

This application claims benefit of U.S. Provisional Application No. 61/048,796, filed Apr. 29, 2008. Application No. 61/048,796, filed Apr. 29, 2008, is hereby incorporated herein by reference in its entirety.

### STATEMENT REGARDING FEDERALLY SPONSORED RESEARCH

This invention was made with government support under grants R03-MH-085677 from the NIH and U54-HG005033 from the NICHD. The government has certain rights in the invention.

### BACKGROUND

Ubiquitin is a highly conserved 76 amino acid protein expressed in all eukaryotic cells. The levels of many intracellular proteins are regulated by a ubiquitin-dependent proteolytic process. This process involves the covalent ligation of ubiquitin to a target protein, resulting in a poly-ubiquitinated target protein which is rapidly detected and degraded by the 26S proteasome.

The ubiquitination of these proteins is mediated by a cascade of enzymatic activity. Ubiquitin is first activated in an ATP-dependent manner by a ubiquitin activating enzyme (E1). The C-terminus of a ubiquitin forms a high energy thiolester bond with E1. The ubiquitin is then passed to a ubiquitin conjugating enzyme (E2; also called ubiquitin carrier protein), also linked to this second enzyme via a thiolester bond. The ubiquitin is finally linked to its target protein to form a terminal isopeptide bond under the guidance of a ubiquitin ligase (E3). In this process, chains of ubiquitin are formed on the target protein, each covalently ligated to the next through the activity of E3.

E1 and E2 are structurally related and well characterized enzymes. There are several species of E2 (at least 25 in mammals), some of which act in preferred pairs with specific E3 enzymes to confer specificity for different target proteins. While the nomenclature for E2 is not standardized across species, investigators in the field have addressed this issue and the skilled artisan can readily identify various E2 proteins, as well as species homologues (See Haas and Siepmann, FASEB J. 11:1257-1268 (1997)).

E3 enzymes contain two separate activities: a ubiquitin ligase activity to conjugate ubiquitin to substrates and form polyubiquitin chains via isopeptide bonds, and a targeting activity to physically bring the ligase and substrate together. Substrate specificity of different E3 enzymes is the major determinant in the selectivity of the ubiquitin-dependent protein degradation process.

Modulators of ubiquitination can be used to upregulate or downregulate specific molecules involved in cellular signal transduction. Disease processes can be treated by such up- or down regulation of signal transducers to enhance or dampen specific cellular responses. This principle has been used in the design of a number of therapeutics, including Phosphodiesterase inhibitors for airway disease and vascular insufficiency, Kinase inhibitors for malignant transformation and Proteasome inhibitors for inflammatory conditions such as arthritis. Thus, due to the importance of ubiquitination in cellular regulation and the wide array of different possible components in ubiquitin-dependent proteolysis, there is a need for a fast and simple means for assaying ubiquitination and identifying modulators thereof.

### BRIEF SUMMARY

In accordance with the purpose of this invention, as embodied and broadly described herein, this invention relates to compositions and methods for assaying ubiquitination. Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows Ubc13-Uev1a dependent ubiquitination reaction.

FIG. 2 shows determination of the activity of Ubc13-Uev1a complex.

FIG. 3 shows determination of optimal time point for the assay.

FIG. 4 shows determination of reaction temperature for optimal TR-FRET signal.

FIG. 5 shows determination of Z′ factor of Ubc13-Uev1a mediated TR-FRET based ubiquitination assay system.

FIG. 6 shows fluorimager analysis of ubiquitination reactions from TR FRET assay methodology.

FIG. 7 shows Ubc13-dependent polyubiquitin chain formation on Uev1a.

FIGS. 8A and 8B show UBC13-catalyzed co-factor (UEV1A versus MMS2) dependent TR-FRET based ubiquitination assay. FIG. 8A shows UBC13-catalyzed, UEV1A dependent ubiquitin chain assembly. TR-FRET measurement from complete reaction mixture consisting of UBC13 in complex with UEV1A was compared to ubiquitination reaction mixture component having UBC13 alone and lacking the co-factor UEV1A. FIG. 8B shows UBC13-catalyzed, MMS2 dependent ubiquitin chain assembly. TR-FRET measurement from complete reaction mixture consisting of UBC13 in complex with MMS2 was compared to ubiquitination reaction mixture component having UBC13 alone and lacking the co-factor MMS2. Reaction components include, Fl-Ub (150 nM), Tb-Ub (10 nM), E1 (12.5 nM), UBC13 (250 nM), UBC13:UEV1A (250 nM each) or UBC13:MMS2 (250 nM each) and ATP regenerating system (1×). Data is represented as mean±SEM. Abscissa α-axis):Reaction incubation time (hr); Ordinate (y-axis):TR-FRET signal expressed as emission ratio (Fl-520 nm/Tb-480 nm). Ratiometric measurements show co-factor dependent increase in TR-FRET signal in a time-dependent fashion. Readings were taken at 1, 3 and 5 hr time points.

FIGS. 9A-9D show kinetics of UBC13-catalyzed, cofactor-mediated (UEV1A or MMS2) ubiquitination reaction. FIGS. 9A and 9C. Kinetic analysis of TR-FRET based ubiquitin polymerization reactions catalyzed by UBC13 and mediated by UEV1A. FIGS. 9B and 9D. Kinetic analysis of TR-FRET based ubiquitination reactions catalyzed by UBC13 in complex with co-factor MMS2. FIGS. 9A and 9B. Initial reaction rates (Vo, nM/min) plotted against substrate concentration ([S], nM, at a 1:15 molar ratio of Tb-Ub:Fl-Ub) yielded Michaelis-Menten kinetic profile. Data is expressed as mean±SEM. Non-linear regression analysis was performed using Prism v. 5.0. FIGS. 9C and 9D. Titration of UBC13-cofactor complex as a Function of TR-FRET signal per unit time. Data is expressed as mean±SEM. Linear regression analysis performed using Prism v. 5.0.

### DETAILED DESCRIPTION

The disclosed method and compositions may be understood more readily by reference to the following detailed description of particular embodiments and the Example included therein and to the Figures and their previous and following description.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the peptide are discussed, each and every combination and permutation of peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Those skilled in the art will recognize, or be able to ascertain using no more than routine experimentation, many equivalents to the specific embodiments of the method and compositions described herein. Such equivalents are intended to be encompassed by the following claims.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

### A. METHODS OF ASSAYING UBIQUITINATION

Disclosed herein are methods for assaying ubiquitination. In some aspects, the method involves measuring ubiquitin polymerization directly where the reaction has occurred, thus obviating the need for target proteins and subsequent analysis such as separating ligated from unligated material in an SDS PAGE procedure. This allows multi-well array analysis and high throughput screening techniques for modulators of ubiquitination activity.

For example, provided herein is a method of identifying a ubiquitination modulator comprising a) combining, under conditions that favor ubiquitination activity ubiquitin, a candidate modulator, ubiquitin activating enzyme (E1) and ubiquitin conjugating enzyme (E2), thereby producing a reaction mixture, and b) measuring the amount of polyubiquitin, whereby a difference in polyubiquitin as compared with a reaction performed in the absence of the candidate modulator indicates that the candidate is a ubiquitination modulator. The reaction mixture can further comprise adenosine tri-phosphate (ATP) and or an ATP regeneration system.

The disclosed methods can comprise assaying ubiquitination without the need for target proteins or an E3 enzyme. Thus, in some aspects of the method, the reaction mixture substantially lacks ubiquitin ligase (E3).

As used herein, “ubiquitination,” “ubiquitinylation,” and grammatical equivalents thereof refer to the binding of ubiquitin to a substrate protein. As used herein, “ubiquitin activating activity”, “ubiquitin activation” and grammatical equivalents thereof refers to the binding of ubiquitin and E1 enzyme. E1 can form a high energy thiolester bond with the ubiquitin. As used herein, “ubiquitin conjugating activity”, “ubiquitin conjugation” and grammatical equivalents thereof refers to the binding of activated ubiquitin with an E2 enzyme.

As used herein, “substrate protein” means a protein to which ubiquitin is bound through the activity of ubiquitination enzymes. In some aspects, the substrate protein is a target protein. By “target protein” herein is meant a protein other than ubiquitin to which ubiquitin is ligated by ubiquitination enzymes. However, in some aspects, no specific target protein is used to measure ubiquitination. In some aspects, the ubiquitination substrate protein is ubiquitin itself, and what is measured is poly-ubiquitin chains. Thus, the method can involve combining ubiquitin and ubiquitination enzymes and measuring ubiquitin polymerization.

In some aspects, the method involves detecting poly-ubiquitination of E2. In some aspects, E2 comprises a combination of a ubiquitin-conjugating enzyme (Ubc) and a ubiquitin E2 variant (Uev). The ubiquitin E2 variant can be, for example, ubiquitin E2 variant 1a (Uev1a) or ubiquitin E2 variant 2 (Mms2 or UBE2V2). In some aspects, diubiquitination of Ubc13 results in poly-ubiquitination of Uev1a. Thus, in some aspects, the method involves detecting poly-ubiquitination of Uev1a. In some aspects, diubiquitination of Ubc13 results in poly-ubiquitination of Mms2. Thus, in some aspects, the method involves detecting poly-ubiquitination of Mms2.

Thus, the disclosed methods can involve ubiquitin polymerization, wherein polyubiquitin chains are formed on ubiquitin conjugating enzymes (E2) in the absence of a ubiquitin ligase (E3) and in the absence of any target protein. Thus, in some aspects of the method, the reaction mixture substantially lacks a non-ubiquitin target protein.

In some aspects, E2 (including Ubc13 and/or Uev1a) is attached to the surface of a reaction vessel, such as the well of a multi-well plate. These aspects facilitate the separation of conjugated ubiquitin from unconjugated ubiquitin. Means of attaching E2 to the surface of a reaction vessel are known and described herein. This aspect allows the ubiquitin conjugation reaction and detection and measurement of polymerized ubiquitin to occur in the same vessel, making the assay useful for high-throughput screening applications.

In some aspects, E2 is free in solution. In these aspects, ubiquitination activity can be monitored using a system that produces a signal which varies with the extent of ubiquitination, such as the fluorescence resonance energy transfer (FRET) system described herein.

Disclosed herein are methods and compositions comprising combining ubiquitin, E1, E2, and optionally a candidate agent, wherein E1 is capable of transferring ubiquitin to the E2. In some aspects, E1 forms a high energy thiolester bond with ubiquitin, thereby “activating” the ubiquitin. In some aspects, ubiquitin can be transferred from E1 to E2. In some aspects, the transfer results in a thiolester bond formed between E2 and ubiquitin. In some aspects, ubiquitin can be transferred from E1 to an E2-ubiquitin conjugate forming a ubiquitin polymer. The reaction mixture can further comprise adenosine tri-phosphate (ATP) and or an ATP regeneration system.

In some aspects, the ubiquitin is labeled, either directly or indirectly, and the amount of label is measured. This allows for easy and rapid detection and measurement of ligated ubiquitin, making the assay useful for high-throughput screening applications. In some aspects, the signal of the label varies with the extent of ubiquitin polymerization.

The proteins of the present method can be recombinant. A “recombinant protein” is a protein made using recombinant techniques, i.e. through the expression of a recombinant nucleic acid as described below. A recombinant protein is distinguished from naturally occurring protein by at least one or more characteristics. For example, the protein can be isolated or purified away from some or all of the proteins and compounds with which it is normally associated in its wild type host, and thus can be substantially pure. For example, an isolated protein is unaccompanied by at least some of the material with which it is normally associated in its natural state, constituting at least about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0% by weight of the total protein in a given sample. A substantially pure protein comprises at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% by weight of the total protein. The definition includes the production of a protein from one organism in a different organism or host cell. Alternatively, the protein can be made at a significantly higher concentration than is normally seen, through the use of an inducible promoter or high expression promoter, such that the protein is made at increased concentration levels. Alternatively, the protein can be in a form not normally found in nature, as in the addition of an epitope tag or amino acid substitutions, insertions and deletions, as discussed below.

1. Components

i. Ubiquitin

The reaction mixture of the disclosed methods can comprise ubiquitin. By “ubiquitin” herein is meant a polypeptide which is ligated to another polypeptide by ubiquitin ligase enzymes. The ubiquitin can be from any species of organism, including a eukaryotic species. In some aspects, the ubiquitin is mammalian. For example, the ubiquitin can be human ubiquitin. Also encompassed by “ubiquitin” are naturally occurring alleles and man-made variants. In some aspects, the ubiquitin has the amino acid sequence of that depicted in accession number P02248 or P62988, which is incorporated herein by reference. In some aspects, the ubiquitin has the amino acid sequence set forth in SEQ ID NO:1. In some aspects, the ubiquitin has an amino acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the amino acid sequence set forth in SEQ ID NO:1.

Ubiquitin proteins of the disclosed methods can be shorter or longer than the amino acid sequence SEQ ID NO:1. Thus, in some aspects, included within the definition of ubiquitin are portions or fragments of the amino acid sequence SEQ ID NO:1. In some aspects herein, fragments of ubiquitin are considered ubiquitin proteins if they are ligated to another polypeptide by ubiquitin ligase enzymes. In addition, ubiquitin can be made longer than the amino acid sequence SEQ ID NO:1; for example, by the addition of tags, the addition of other fusion sequences, or the elucidation of additional coding and non-coding sequences. As described below, the fusion of a ubiquitin peptide to a tag, such as a fluorescent peptide, is disclosed herein.

ii. E1 Ubiquitin Enzyme

The reaction mixture of the disclosed methods can comprise E1 enzyme. By “E1” is meant a polypeptide which can form a high energy thiolester bond with a ubiquitin thereby activating the ubiquitin. The E1 can be from any species of organism, including a eukaryotic species. In some aspects, the E1 is mammalian. For example, the E1 can be human E1. Also encompassed by “E1” are naturally occurring alleles and man-made variants.

E1 proteins useful in the disclosed methods include those having the amino acid sequence of the polypeptide having accession numbers A38564, S23770, AAA61246, P22314, CAA40296 and BAA33144, incorporated herein by reference. E1 is commercially available from Affiniti Research Products (Exeter, U.K.). Nucleic acids that can be used for producing E1 proteins for the method include, but are not limited to, those disclosed by accession numbers M58028, X56976 and AB012190, incorporated herein by reference.

In some aspects, the E1 of the disclosed methods is human E1. Thus, E1 proteins useful in the disclosed methods include, but are not limited to, those having the amino acid sequences disclosed in accession numbers NP_{—}695012 (SEQ ID NO:4), which is incorporated herein by reference. In some aspects, the E1 has the amino acid sequence set forth in SEQ ID NO:4. In some aspects, the E1 has an amino acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the amino acid sequence set forth in SEQ ID NO:4.

The heterodimeric partner of human E1 known as UBA2 (accession number NP_{—}005490) specific for the SUMO family of ubiquitin-like proteins can be included as a control. Thus, E1 proteins useful as controls in the disclosed methods include, but are not limited to, those having the amino acid sequences disclosed in accession numbers NP_{—}005490 (SEQ ID NO:5), which is incorporated herein by reference. In some aspects, the E1 has the amino acid sequence set forth in SEQ ID NO:5. In some aspects, the E1 has an amino acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the amino acid sequence set forth in SEQ ID NO:5.

iii. E2 Ubiquitin-Conjugating Enzyme (Ubc) and Ubiquitin E2 Variant (UEV)

The reaction mixture of the disclosed methods can comprise E2 enzyme. By “E2” is meant a polypeptide or polypeptides which can form a high energy thiolester bond with an activated ubiquitin and bind a ubiquitin ligase. The E2 can be from any species of organism, including a eukaryotic species. In some aspects, the E2 is mammalian. For example, the E2 can be human E2. Also encompassed by “E2” are naturally occurring alleles and man-made variants.

“E2” as used herein refers to canonical E2 ubiquitin-conjugating enzymes (Ubc), ubiquitin E2 variants (Uev), or combinations thereof. Thus, the compositions of the disclosed methods can comprise a ubiquitin-conjugating enzyme (Ubc) and a ubiquitin E2 variant (Uev). In some aspects, the method involves detecting poly-ubiquitination of Uev1a.

The skilled artisan will appreciate that many different Ubc proteins and isozymes are known in the field and can be used in the present methods, provided that the Ubc has ubiquitin conjugating activity. The Ubc can be human Ubc. In some aspects, the Ubc is one of Ubc5 (Ubch5, Ubch5c), Ubc3 (Ubch3), Ubc4 (Ubch4) and UbcX (Ubc10, Ubch10). Thus, Ubc proteins useful in the disclosed methods include, but are not limited to, those having the amino acid sequences disclosed in accession numbers AAC37534, P49427, CAA82525, AAA58466, AAC41750, P51669, AAA91460, AAA91461, CAA63538, AAC50633, P27924, AAB36017, Q16763, AAB86433, AAC26141, CAA04156, BAA11675, Q16781, NP_{—}003333, BAB18652, AAH00468, CAC16955, CAB76865, CAB76864, NP_{—}05536, O00762, XP_{—}009804, XP_{—}009488, XP_{—}006823, XP_{—}006343, XP_{—}005934, XP_{—}002869, XP_{—}003400, XP_{—}009365, XP.sub.-010361, XP_{—}004699, XP_{—}004019, O14933, P27924, P50550, P52485, P51668, P51669, P49459, P37286, P23567, P56554, CAB45853, NP003331, NP003330, NP003329, P49427, AAB53362, NP008950, XP009488, and AAC41750, each of which is incorporated herein by reference. In some aspects, nucleic acids which can be used to make Ubc include, but are not limited to, those nucleic acids having sequences disclosed in accession numbers L2205, Z29328, M92670, L40146, U39317, U39318, X92962, U58522, S81003, AF031141, AF075599, AJ000519, XM009488, NM007019, U73379, L40146 and D83004, each of which is incorporated herein by reference. As described above, variants of these and other Ubc encoding nucleic acids can also be used to make variant Ubc proteins.

In some aspects, the Ubc of the disclosed methods is Ubc13. Thus, Ubc proteins useful in the disclosed methods include, but are not limited to, those having the amino acid sequences disclosed in accession numbers NP_{—}003339 (SEQ ID NO:2).

In some aspects, the Ubc13 has the amino acid sequence set forth in SEQ ID NO:2. In some aspects, the Ubc13 has an amino acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the amino acid sequence set forth in SEQ ID NO:2.

Ubc13 proteins of the disclosed methods can be shorter or longer than the amino acid sequence SEQ ID NO:2. Thus, in some aspects, included within the definition of Ubc13 are portions or fragments of the amino acid sequence SEQ ID NO:2. In addition, Ubc13 can be made longer than the amino acid sequence SEQ ID NO:2; for example, by the addition of tags, the addition of other fusion sequences, or the elucidation of additional coding and non-coding sequences.

Ubc13 requires the presence of a Uev for polyubiquitination. Uevs are similar to ubiquitin-conjugating enzyme (Ubc; canonical E2) in both sequence and structure, but the lack of a catalytic cysteine residue renders them incapable of forming a thiol-ester linkage with ubiquitin. Divergent activities of mammalian Ubc13 rely on its pairing with either of two Uevs, Uev1A or Mms2. Thus, in some aspects of the disclosed methods, the E2 of the disclosed method comprises a combination of a Ubc and a Uev. The Uev can be a human Uev. In some aspects, the Uev of the disclosed methods is Uev1a or Mms2. Thus, Uev proteins useful in the disclosed methods include, but are not limited to, those having the amino acid sequences disclosed in accession numbers NP_{—}068823 (SEQ ID NO:3), NP_{—}071887 (SEQ ID NO:6), NP_{—}001027459 (SEQ ID NO:7), or NM_{—}003341.1 (SEQ ID NO:15).

In some aspects, the Uev is an isoform of hUev1a. In some aspects, the isoform differs from hUev1a in the 5′ UTR of the nucleic acid but encodes the same amino acid. In some aspects, the isoform differs in the 5′ UTR and/or coding region. For example, the isoform can lack an alternate in-frame exon. The resulting isoform protein can be shorter and have a distinct N-terminus, compared to variant 1. In some aspects, the isoform differs in the 5′ UTR and coding region compared to variant 1. The resulting isoform is shorter and has a distinct N-terminus compared to hUev1a.

Thus, in some aspects, the Uev has the amino acid sequence set forth in SEQ ID NO:3, SEQ ID NO:6, SEQ ID NO:7, or SEQ ID NO:15. In some aspects, the Uev has an amino acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the amino acid sequence set forth in SEQ ID NO:3 or SEQ ID NO:15.

Uev proteins of the disclosed methods can be shorter or longer than the amino acid sequence SEQ ID NO:3, SEQ ID NO:6, SEQ ID NO:7, or SEQ ID NO:15. Thus, in some aspects, included within the definition of Uev are portions or fragments of the amino acid sequence SEQ ID NO:3, SEQ ID NO:6, SEQ ID NO:7, or SEQ ID NO:15. In addition, Uev can be made longer than the amino acid sequence SEQ ID NO:3, SEQ ID NO:6, SEQ ID NO:7, or SEQ ID NO:15; for example, by the addition of tags, the addition of other fusion sequences, or the elucidation of additional coding and non-coding sequences.

iv. Adenosine Tri-Phosphate (ATP)

The reaction mixture of the disclosed methods can comprise Adenosine Tri-Phosphate (ATP). The reaction mixture of the disclosed methods can further comprise a ATP regenerating system.

Thus, the reaction mixture of the disclosed methods can comprise creatine kinase and phosphocreatine. Creatine kinase (CK), also known as phosphocreatine kinase or creatine phosphokinase (CPK) is an enzyme (EC 2.7.3.2) catalyses the conversion of creatine to phosphocreatine, consuming adenosine triphosphate (ATP) and generating adenosine diphosphate (ADP).

ATP+creatine [symbol] ADP+phosphocreatine

Phosphocreatine, also known as creatine phosphate or Pcr, is a phosphorylated creatine molecule that is an important energy store in skeletal muscle. It is used to anaerobically generate ATP from ADP, forming creatine for the 2 to 7 seconds following an intense effort. It does that by donating a phosphate group and this reaction is catalyzed by creatine. This reaction is reversible and it therefore acts as a spatial and temporal buffer of ATP concentration. Thus, creatine kinase and phosphocreatine can be used in the reaction mixture to maintain ATP levels.

v. Candidate Agents

The reaction mixture of the disclosed methods can comprise a candidate modulator. By “candidate agent,” “candidate modulator” or grammatical equivalents herein is meant any molecule, e.g. proteins (which herein includes proteins, polypeptides, and peptides), small organic or inorganic molecules, polysaccharides, polynucleotides, etc. which are to be tested for ubiquitination modulator activity.

In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the method. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods. In addition, those skilled in the art of drug discovery and development readily understand that methods for dereplication (e.g., taxonomic dereplication, biological dereplication, and chemical dereplication, or any combination thereof) or the elimination of replicates or repeats of materials already known can be employed whenever possible.

When a crude extract is found to have a desired activity, further fractionation of the positive lead extract can be performed to isolate chemical constituents responsible for the observed effect. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value can be subsequently analyzed using animal models for diseases or conditions, such as those disclosed herein.

Candidate agents encompass numerous chemical classes, including organic molecules, e.g., small organic compounds having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, for example, at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

In some aspects, the candidate agents are naturally occurring proteins or fragments of naturally occurring proteins. Thus, for example, cellular extracts containing proteins, or random or directed digests of proteinaceous cellular extracts, can be used. In this way libraries of procaryotic and eucaryotic proteins can be made for screening using the methods herein. The libraries can be bacterial, fungal, viral, and vertebrate proteins, and human proteins.

Once made, the compositions find use in a number of applications, including, but not limited to, screens for modulators of ubiquitination. By “modulator” is meant a compound which can increase or decrease ubiquitination. The skilled artisan will appreciate that modulators of ubiquitination can affect enzyme activity, enzyme interaction with ubiquitin.

In some aspects, candidate modulators are synthetic compounds. Any number of techniques are available for the random and directed synthesis of a wide variety of organic compounds and biomolecules, including expression of randomized oligonucleotides. See for example WO 94/24314, hereby expressly incorporated by reference, which discusses methods for generating new compounds, including random chemistry methods as well as enzymatic methods. As described in WO 94/24314, one of the advantages of the present method is that it is not necessary to characterize the candidate modulator prior to the assay; only candidate modulators that increase or decease ubiquitin ligase activity need be identified. In addition, as is known in the art, coding tags using split synthesis reactions can be done to essentially identify the chemical moieties tested.

In some aspects, the candidate modulators are peptides of about 2, 3, 4, 5, 6, 7, 8, 9, 10, 11, 12, 13, 14, 15, 16, 17, 18, 19, 20, 25, 30, 35, 40, 45, 50 or more amino acids in length. The peptides can be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids) are chemically synthesized, they can incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In some aspects, the library is fully randomized, with no sequence preferences or constants at any position. In some aspects, the library is biased. That is, some positions within the sequence are either held constant, or are selected from a limited number of possibilities. For example, the nucleotides or amino acid residues can be randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, etc., or to purines, etc.

In some aspects, the candidate modulators are organic moieties. In these aspects, as is generally described in WO 94/24314, candidate agents are synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions can be done on the moieties to form new substrates or candidate agents which can then be tested using the present method.

As will be appreciated by those in the art, it is possible to screen more than one type of candidate modulator at a time. Thus, the library of candidate modulators used can include only one type of agent (e.g., peptides), or multiple types (e.g., peptides and organic agents). The assay of several candidates at one time is further discussed below.

vi. Nucleic Acids

Disclosed are nucleic acids encoding each of the amino acids disclosed herein.

For example, the nucleic acid encoding E1 of the disclosed methods can comprise the nucleic acid sequence SEQ ID NO:12.

For example, the nucleic acid encoding Ubc13 of the disclosed methods can comprise the nucleic acid sequence SEQ ID NO:13.

For example, the nucleic acid encoding Uev1a of the disclosed methods can comprise the nucleic acid sequence SEQ ID NO:8, SEQ ID NO:9, SEQ ID NO:10, or SEQ ID NO:11. The nucleic acid encoding Mms2 of the disclosed methods can comprise the nucleic acid sequence SEQ ID NO:14.

The disclosed nucleic acids can be made up of for example, nucleotides, nucleotide analogs, or nucleotide substitutes. Non-limiting examples of these and other molecules are discussed herein. It is understood that for example, when a vector is expressed in a cell, the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a. Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b. Sequences

There are a variety of sequences related to the protein disclosed herein. The sequences for the human analogs of these genes, as well as other analogs, and alleles of these genes, and splice variants and other types of variants, are available in a variety of protein and gene databases, including Genbank. Those sequences available at the time of filing this application at Genbank are herein incorporated by reference in their entireties as well as for individual subsequences contained therein. Genbank can be accessed at http://www.ncbi.nih.gov/entrez/query.fcgi. Those of skill in the art understand how to resolve sequence discrepancies and differences and to adjust the compositions and methods relating to a particular sequence to other related sequences. Primers and/or probes can be designed for any given sequence given the information disclosed herein and known in the art.

vii. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods can differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

2. Assay Conditions

The disclosed methods comprise combining ubiquitin with other components. By “combining” is meant the addition of the various components into a receptacle under conditions in which ubiquitination can take place. In some aspects, the receptacle is a well of a 96 well plate or other commercially available multiwell plate. In some aspects, the receptacle is the reaction vessel of a FACS machine. Other receptacles useful in the present methods include, but are not limited to 384 well plates and 1536 well plates. Still other receptacles useful in the present methods will be apparent to the skilled artisan. The addition of the components can be sequential or in a predetermined order or grouping, as long as the conditions amenable to ubiquitination are obtained. Such conditions are well known in the art, and further guidance is provided below.

The components of the present compositions can be combined in varying amounts. In some aspects, the reaction mixture comprises ubiquitin at a final concentration of about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400 nM.

In some aspects, the reaction mixture comprises tag1-ubiquitin and tag2-ubiquitin. In some aspects, the reaction mixture comprises tag1-ubiquitin at a final concentration of about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400 nM and tag2-ubiquitin at a final concentration of about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400 nM. In some aspects, the reaction mixture comprises tag1-ubiquitin and tag2-ubiquitin at a ratio of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. For example, in some aspects, the reaction mixture comprises final concentrations of tag1-ubiquitin at about 10 nM and tag2-ubiquitin at about 150 nM. As exemplified herein, the reaction mixture can comprise 10 nM terbium-labeled ubiquitin and 150 nM fluorescein-labeled ubiquitin. Other such examples are determinable using routine experimentation to identify optimal concentrations.

In some aspects, the reaction mixture comprises E1 at a final concentration of about 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400 nM. The term “about” is meant to include amounts between two values. Thus, “about 12, 13” includes 12.1, 12.2, 12.3, 12.4, 12.5, 12.6, 12.7, 12.8, 12.9.

In some aspects, the reaction mixture comprises E2 at a final concentration of about 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400 nM.

In some aspects, the E2 of the reaction mixture comprises Ubc13 and Uev1a. In some aspects, the reaction mixture comprises Ubc13 at a final concentration of about 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400 nM and Uev1a at a final concentration of about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400 nM. In some aspects, the reaction mixture comprises Ubc13 and Uev1a at a ratio of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. For example, in some aspects, the reaction mixture comprises final concentrations of Ubc13 at about 250 nM and Uev1a at about 250 nM. Other such examples are determinable using routine experimentation to identify optimal concentrations.

In some aspects, the reaction mixture comprises ATP at a final concentration of about 0.01, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, and 3.0 mM. Other such examples are determinable using routine experimentation to identify optimal concentrations.

In some aspects, the reaction mixture comprises phosphocreatine at a final concentration of about 0.01, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10 mM. Other such examples are determinable using routine experimentation to identify optimal concentrations.

In some aspects, the reaction mixture comprises creatine kinase (creatine phosphokinase) at a final concentration of about 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0 units/μl. Other such examples are determinable using routine experimentation to identify optimal concentrations.

In some aspects, the reaction mixture comprises MgCl₂ at a final concentration of about 0.01, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10 nM. Other such examples are determinable using routine experimentation to identify optimal concentrations.

The components in the reaction mixture of the disclosed methods can be combined under reaction conditions that favor ubiquitination activity. Generally, this can be physiological conditions. Incubations can be performed at any temperature which facilitates optimal activity, including at about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40° C. Incubation periods can be selected for optimum activity, but can also be optimized to facilitate rapid high through put screening. For example, the incubation period can be about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0 hours or longer.

A variety of other reagents can be included in the compositions. These include reagents like salts, solvents, buffers, neutral proteins, e.g. albumin, detergents, etc. which can be used to facilitate optimal ubiquitination enzyme activity and/or reduce non-specific or background interactions. Also reagents that otherwise improve the efficiency of the assay, such as protease inhibitors, nuclease inhibitors, anti-microbial agents, etc., can be used.

The mixture of components can be added in any order that promotes ubiquitination or optimizes identification of candidate modulator effects. In some aspects, ubiquitin is provided in a reaction buffer solution, followed by addition of the ubiquitination enzymes. In some aspects, ubiquitin is provided in a reaction buffer solution, a candidate modulator is then added, followed by addition of the ubiquitination enzymes.

In some aspects, none of the ubiquitination enzymes are bound to a substrate. In these aspects, the composition can comprise tag1-ubiquitin, tag2-ubiquitin, E1, and E2 (e.g., Ubc13/Uev1a). In some aspects, tag1 and tag2 are labels, such as fluorescent labels. In some aspects, tag1 and tag2 constitute a FRET pair. In these aspects, ubiquitination is measured by measuring the fluorescent emission spectrum. This measuring can be continuous or at one or more times following the combination of the components. Alteration in the fluorescent emission spectrum of the combination as compared with unligated ubiquitin indicates the amount of ubiquitination. The skilled artisan can appreciate that in this aspect, alteration in the fluorescent emission spectrum results from ubiquitin bearing different members of the FRET pair being brought into close proximity in the formation of poly-ubiquitin.

In some aspects, multiple assays are performed simultaneously in a high throughput screening system. In these aspects, multiple assays can be performed in multiple receptacles, such as the wells of a 96 well plate or other multi-well plate. As will be appreciated by one of skill in the art, such a system can be applied to the assay of multiple candidate modulators and multiple combination of components. In some aspects, the present method is used in a high throughput screening system for simultaneously testing the effect of individual candidate modulators.

3. Detection

Ubiquitin polymerization can be detected using routine method. In some aspects, one or more components, such as the ubiquitin, of the present methods comprise a tag. By “tag” is meant an attached molecule or molecules useful for the identification or isolation of the attached component. Components having a tag are referred to as “tag-X”, wherein X is the component. For example, a ubiquitin comprising a tag is referred to herein as “tag-ubiquitin.” Moreover, reference to a component is also a reference to that component attached to a tag. For example, reference to an E1 enzyme is also a reference to tag-E1, such as His-E1, which can be used, for example, to isolate, purify, or identify the E1 enzyme.

The tag can be covalently bound to the attached component. When more than one component of a combination has a tag, the tags can be numbered for identification, for example “tag1-ubiquitin”. Components can comprise more than one tag, in which case each tag can be numbered, for example “tag1,2-ubiquitin”. Exemplary tags include, but are not limited to, a label, a partner of a binding pair, and a surface substrate binding molecule. As will be evident to the skilled artisan, many molecules can find use as more than one type of tag, depending upon how the tag is used.

Thus, provided is a method of identifying a ubiquitination modulator comprising: a) combining, under conditions that favor ubiquitination activity tag1-ubiquitin, tag2-ubiquitin, a candidate modulator, ubiquitin activating enzyme (E1), and ubiquitin conjugating enzyme (E2), thereby producing a reaction mixture; and b) measuring the amount of tag1-ubiquitin bound to said tag2-ubiquitin in said reaction mixture, whereby a difference in bound ubiquitin as compared with a reaction performed in the absence of the candidate modulator indicates that the candidate is a ubiquitination modulator.

In some aspects of the method, the reaction mixture substantially lacks ubiquitin ligase (E3). In some aspects, the ubiquitin conjugating enzyme (E2) comprises Ubiquitin conjugating enzyme 13 (Ubc13). In some aspects, the ubiquitin conjugating enzyme (E2) comprises Ubiquitin E2 variant 1a (Uev1a). In some aspects, the ubiquitin conjugating enzyme (E2) comprises Ubc13 and Uev1a. In some aspects, tag1 and tag2 are fluorescent labels constituting a fluorescence resonance energy transfer (FRET) pair. In some aspects, said combining and measuring is performed in a multi-well plate comprising a surface substrate comprising nickel.

By “label” is meant a molecule that can be directly (i.e., a primary label) or indirectly (i.e., a secondary label) detected; for example a label can be visualized and/or measured or otherwise identified so that its presence or absence can be known. As will be appreciated by those in the art, the manner in which this is done can depend on the label. Exemplary labels include, but are not limited to, fluorescent labels, label enzymes and radioisotopes.

By “fluorescent label” is meant any molecule that an be detected via its inherent fluorescent properties. Suitable fluorescent labels include, but are not limited to 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy 492/515; Bodipy 493/503; Bodipy 500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson-; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydrorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydrorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyde Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type′ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (Pl); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

By “label enzyme” is meant an enzyme which can be reacted in the presence of a label enzyme substrate which produces a detectable product. Suitable label enzymes for use in the present methods include but are not limited to, horseradish peroxidase, alkaline phosphatase and glucose oxidase. Methods for the use of such substrates are well known in the art. The presence of the label enzyme is generally revealed through the enzyme's catalysis of a reaction with a label enzyme substrate, producing an identifiable product. Such products can be opaque, such as the reaction of horseradish peroxidase with tetramethyl benzedine, and can have a variety of colors. Other label enzyme substrates, such as Luminol (available from Pierce Chemical Co.), have been developed that produce fluorescent reaction products. Methods for identifying label enzymes with label enzyme substrates are well known in the art and many commercial kits are available. Examples and methods for the use of various label enzymes are described in Savage et al., Previews 247:6-9 (1998), Young, J. Virol. Methods 24:227-236 (1989), which are each hereby incorporated by reference in their entirety.

i. FRET

In some instances, multiple fluorescent labels are used. In some aspects, at least two fluorescent labels are used which are members of a Fluorescence (Förster) Resonance Energy Transfer (FRET) pair. FRET refers to an energy transfer mechanism between two chromophores. A donor chromophore in its excited state can transfer energy by a nonradiative, long-range dipole-dipole coupling mechanism to an acceptor chromophore in close proximity (typically <10 nm).

A FRET pair consists of a donor fluorophore and an acceptor fluorophore. The fluorescence emission spectrum of the donor and the fluorescence absorption spectrum of the acceptor must overlap, and the two molecules must be in close proximity. The distance between donor and acceptor at which 50% of donors are deactivated (transfer energy to the acceptor) is defined by the Förster radius (R_{O}), which is typically 10-100 Å. Changes in the fluorescence emission spectrum comprising FRET pairs can be detected, indicating changes in the number of that are in close proximity (i.e., within 100 Å of each other). This will typically result from the binding or dissociation of two molecules, one of which is labeled with a FRET donor and the other of which is labeled with a FRET acceptor, wherein such binding brings the FRET pair in close proximity. Binding of such molecules can result in an increased fluorescence emission of the acceptor and/or quenching of the fluorescence emission of the donor.

An example of a FRET pair for biological use is a cyan fluorescent protein (CFP)-yellow fluorescent protein (YFP) pair. Both are color variants of green fluorescent protein (GFP). While labeling with organic fluorescent dyes requires troublesome processes of purification, chemical modification, and intracellular injection of a host protein, GFP variants can be easily attached to a host protein by genetic engineering.

Other FRET pairs (donor/acceptor) useful in the present methods include, but are not limited to, EDANS/fluorescien, IAEDANS/fluorescein, fluorescein/tetramethylrhodamine, fluorescein/LC Red 640, fluorescein/Cy 5, fluorescein/Cy 5.5 and fluorescein/LC Red 705.

In another aspect of FRET, a fluorescent donor molecule and a nonfluorescent acceptor molecule (“quencher”) can be employed. In this application, fluorescent emission of the donor can increase when quencher is displaced from close proximity to the donor and fluorescent emission can decrease when the quencher is brought into close proximity to the donor. Useful quenchers include, but are not limited to, DABCYL, QSY 7 and QSY 33. Useful fluorescent donor/quencher pairs include, but are not limited to EDANS/DABCYL, Texas Red/DABCYL, BODIPY/DABCYL, Lucifer yellow/DABCYL, coumarin/DABCYL and fluorescein/QSY 7 dye.

Many compounds and proteins present in biological fluids or serum are naturally fluorescent, and the use of conventional, prompt fluorophores leads to serious limitations in assay sensitivity. The use of long-lived fluorophores combined with time-resolved detection (a delay between excitation and emission detection) minimizes prompt fluorescence interferences. Time-resolved fluorometry (TRF) takes advantage of the unique properties of the rare earth elements called lanthanides. The commonly used lanthanides in TRF assays are samarium (Sm), europium (Eu), terbium (Tb), and dysprosium (Dy). Because of their specific photophysical and spectral properties, complexes of rare earth ions are of major interest for fluorescence applications in biology. Specifically, they have large Stoke's shifts and extremely long emission half-lives (from μsec to msec) when compared to more traditional fluorophores. Thus, in some aspects the FRET pairs of the disclosed method are terbium and fluorescein.

It is difficult to generate fluorescence of lanthanide ions by direct excitation, because of the ions' poor ability to absorb light. Lanthanides can therefore be complexed with organic moieties that harvest light and transfer it to the lanthanide through intramolecular, non-radiative processes. Rare earth chelates and cryptates are examples of light-harvesting devices. The collected energy is transferred to the rare earth ion, which then emits its characteristic long-lived fluorescence.

Commercial systems are available from Wallac, Oy, Turku, Finland and Packard Instrument Company, Meriden, USA, which use lanthanide chelates as the donor label and dyes from the phycobiliprotein class e.g. allophycocyanin as the acceptor label. The lanthanide chelates have a luminescence lifetime in a range up to several milliseconds i.e. the acceptor emission can be observed for a corresponding length of time. Hence the energy released by lanthanide chelates is usually measured in a time window between 400-600 microseconds. This also inevitably means that there are also relatively long dead times. The stability of the lanthanide chelates is reduced under certain test conditions; thus for example a re-chelation can occur when complexing agents such as EDTA (ethylene-di-amino-tetra-acetic acid) are added.

U.S. Pat. No. 5,998,146 is incorporated herein by reference for the teaching of lanthanide chelate complexes, such as europium and terbium complexes, combined with fluorophores or quenchers. Ruthenium complexes can also be used for time-resolved fluorescent measurement where lumazine is used as the energy donor and a ruthenium complex is used as the energy acceptor. The dye “reactive blue” can also used as the resonance energy acceptor for ruthenium complexes. Reactive blue suppresses the fluorescence emitted by the ruthenium complex and hence the quantification is based on the suppressed fluorescence signal which was originally emitted by the ruthenium complex. Ruthenium complex known as “Fair Oaks Red™” can be used as the energy donor, and fast green or light green yellowish can be used as acceptors for ruthenium complexes.

Also disclosed are detection methods which additionally utilize a time-delayed measurement of the signal from a FRET system. The principle of time-resolved FRET measurements is essentially based on selecting a measuring window such that interfering background fluorescence, e.g., due to interfering substances in the sample, is not co-detected, but rather only the fluorescence generated or suppressed by the energy transfer is measured. The resulting fluorescence of the TR-FRET system can be determined by means of appropriate measuring devices. Such time-resolved detection systems use for example pulsed laser diodes, light emitting diodes (LEDs) or pulsed dye lasers as the excitation light source. The measurement occurs after an appropriate time delay i.e. after the interfering background signals have decayed

FRET systems based on metallic complexes as energy donors and dyes from the class of phycobiliproteins as energy acceptors are known in the art. Established commercial systems (e.g. from Wallac, OY or Cis Bio Packard) use a FRET pair consisting of a lanthanide chelate as the metallic complex and a phycobiliprotein. The advantageous properties of the lanthanide-chelate complexes in particular of europium or terbium complexes are known and can be used in combination with quenchers as well as in combination with fluorophores.

TR-FRET unites TRF (Time-Resolved Fluorescence) and FRET (Fluorescence Resonance Energy Transfer) principles. This combination brings together the low background benefits of TRF with the homogeneous assay format of FRET. This powerful combination provides significant benefits to drug discovery researchers including assay flexibility, reliability, increased assay sensitivity, higher throughput and fewer false positive/false negative results. HTRF® is a TR-FRET based technology that uses the principles of both TRF and FRET. The HTRF® donor fluorophore is either Europium cryptate (Eu3+ cryptate) or Lumi4™-Tb (Tb2+ cryptate). Both donors have the long-lived emissions of lanthanides coupled with the stability of cryptate encapsulation. XL665, a modified allophycocyanin, is the HTRF® primary acceptor fluorophore.

When these two fluorophores are brought together by a biomolecular interaction, a portion of the energy captured by the Cryptate during excitation is released through fluorescence emission at 620 nm, while the remaining energy is transferred to XL665. This energy is then released by XL665 as specific fluorescence at 665 nm. Light at 665 nm is emitted only through FRET with Europium. Because Europium Cryptate is present in the assay, light at 620 nm is detected even when the biomolecular interaction does not bring XL665 within close proximity.

ii. Binding Pairs

In addition, labels can be indirectly detected, such as wherein the tag is a partner of a binding pair. By “partner of a binding pair” is meant one of a first and a second moiety, wherein said first and said second moiety have a specific binding affinity for each other. Suitable binding pairs for use in the method include, but are not limited to, antigens/antibodies (for example, digoxigenin/anti-digoxigenin, dinitrophenyl (DNP)/anti-DNP, dansyl-X-anti-dansyl, Fluorescein/anti-fluorescein, lucifer yellow/anti-lucifer yellow, and rhodamine anti-rhodamine), biotin/avid (or biotin/streptavidin) and calmodulin binding protein (CBP)/calmodulin. Other suitable binding pairs include polypeptides such as the FLAG-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)] and the antibodies each thereto. Generally, in some aspects, the smaller of the binding pair partners serves as the tag, as steric considerations in ubiquitination can be important. As will be appreciated by those in the art, binding pair partners can be used in applications other than for labeling.

As will be appreciated by those in the art, a partner of one binding pair can also be a partner of another binding pair. For example, an antigen (first moiety) can bind to a first antibody (second moiety) which can, in turn, be an antigen for a second antibody (third moiety). It will be further appreciated that such a circumstance allows indirect binding of a first moiety and a third moiety via an intermediary second moiety that is a binding pair partner to each.

As will be appreciated by those in the art, a partner of a binding pair can comprise a label. It will further be appreciated that this allows for a tag to be indirectly labeled upon the binding of a binding partner comprising a label. Attaching a label to a tag which is a partner of a binding pair, as just described, is referred to herein as “indirect labeling”.

By “surface substrate binding molecule” and grammatical equivalents thereof is meant a molecule have binding affinity for a specific surface substrate, which substrate is generally a member of a binding pair applied, incorporated or otherwise attached to a surface. Suitable surface substrate binding molecules and their surface substrates include, but are not limited to poly-histidine (poly-his) or poly-histidine-glycine (poly-his-gly) tags and Nickel substrate; the Glutathione-S Transferase tag and its antibody substrate (available from Pierce Chemical); the flu HA tag polypeptide and its antibody 12CA5 substrate [Field et al., Mol. Cell. Biol., 8:2159-2165 (1988)]; the c-myc tag and the 8F9, 3C7, 6E10, G4, B7 and 9E10 antibody substrates thereto [Evan et al., Molecular and Cellular Biology, 5:3610-3616 (1985)]; and the Herpes Simplex virus glycoprotein D (gD) tag and its antibody substrate [Paborsky et al., Protein Engineering, 3(6):547-553 (1990)]. In general, surface binding substrate molecules useful in the present methods include, but are not limited to, polyhistidine structures (His-tags) that bind nickel substrates, antigens that bind to surface substrates comprising antibody, haptens that bind to avidin substrate (e.g., biotin) and CBP that binds to surface substrate comprising calmodulin.

Biotinylation of target molecules and substrates is well known, for example, a large number of biotinylation agents are known, including amine-reactive and thiol-reactive agents, for the biotinylation of proteins, nucleic acids, carbohydrates, carboxylic acids; see chapter 4, Molecular Probes Catalog, Haugland, 6th Ed. 1996, hereby incorporated by reference. A biotinylated substrate can be attached to a biotinylated component via avidin or streptavidin. Similarly, a large number of haptenylation reagents are also known.

Methods for labeling of proteins with radioisotopes are known in the art. For example, such methods are found in Ohta et al., Molec. Cell 3:535-541 (1999), which is hereby incorporated by reference in its entirety. By “radioisotope” is meant any radioactive molecule. Suitable radioisotopes for use in the method include, but are not limited to ¹⁴C, ³H, ³²P, ³³P, ³⁵S, ¹²⁵I, and ¹³¹I. The use of radioisotopes as labels is well known in the art.

The functionalization of labels with chemically reactive groups such as thiols, amines, carboxyls, etc. is generally known in the art. In some aspects, the tag is functionalized to facilitate covalent attachment.

iii. Tag Attachment

The covalent attachment of the tag can be either direct or via a linker. In some aspects, the linker is a relatively short coupling moiety, that is used to attach the molecules. A coupling moiety can be synthesized directly onto a component of the method, ubiquitin for example, and contains at least one functional group to facilitate attachment of the tag. Alternatively, the coupling moiety can have at least two functional groups, which are used to attach a functionalized component to a functionalized tag, for example. In some aspects, the linker is a polymer. In this aspect, covalent attachment is accomplished either directly, or through the use of coupling moieties from the component or tag to the polymer. In some aspects, the covalent attachment is direct, that is, no linker is used. In this aspect, the component can contain a functional group such as a carboxylic acid which is used for direct attachment to the functionalized tag. It should be understood that the component and tag can be attached in a variety of ways, including those listed above. What is important is that manner of attachment does not significantly alter the functionality of the component. For example, in tag-ubiquitin, the tag should be attached in such a manner as to allow the ubiquitin to be covalently bound to other ubiquitin to form polyubiquitin chains. As will be appreciated by those in the art, the above description of covalent attachment of a label and ubiquitin applies equally to the attachment of virtually any two molecules of the present disclosure.

In some aspects, the tag is functionalized to facilitate covalent attachment. Thus, a wide variety of tags are commercially available which contain functional groups, including, but not limited to, isothiocyanate groups, amino groups, haloacetyl groups, maleimides, succinimidyl esters, and sulfonyl halides, all of which can be used to covalently attach the tag to a second molecule, as is described herein. The choice of the functional group of the tag can depend on the site of attachment to either a linker, as outlined above or a component of the method. Thus, for example, for direct linkage to a carboxylic acid group of a ubiquitin, amino modified or hydrazine modified tags can be used for coupling via carbodiimide chemistry, for example using 1-ethyl-3-(3-dimethylaminopropyl)-carbodiimi-de (EDAC) as is known in the art (see Set 9 and Set 11 of the Molecular Probes Catalog, supra; see also the Pierce 1994 Catalog and Handbook, pages T-155 to T-200, both of which are hereby incorporated by reference). In some aspects, the carbodiimide is first attached to the tag, such as is commercially available for many of the tags described herein.

In some aspects, ubiquitin is in the form of tag-ubiquitin. In some aspects, ubiquitin is in the form of tag-ubiquitin, wherein, tag is a partner of a binding pair. In some aspects, ubiquitin is in the form of tag-ubiquitin, wherein the tag is a fluorescent label. In some aspects, ubiquitin is in the form of tag1-ubiquitin and tag2-ubiquitin, wherein tag1 and tag2 are the members of a FRET pair. In some aspects, ubiquitin is in the form of tag1-ubiquitin and tag2-ubiquitin, wherein tag1 is a fluorescent label and tag2 is a quencher of the fluorescent label. In some aspects, when tag1-ubiquitin and tag2-ubiquitin are polymerized, tag1 and tag2 are within 100 Å, 90 Å, 80 Å, 70 Å, 60 Å, 50 Å, 40 Å, 30 Å or less.

It is important to remember that ubiquitin is ligated protein by its terminal carboxyl group to a lysine residues on other ubiquitin. Therefore, attachment of labels or other tags should not interfere with either of these active groups on the ubiquitin. Amino acids can be added to the sequence of protein, through means well known in the art and described herein, for the express purpose of providing a point of attachment for a label. In some aspects, one or more amino acids are added to the sequence of a component for attaching a tag thereto. In some aspects, the amino acid to which a tag or label is attached is cysteine.

### B. METHODS OF MAKING THE COMPOSITIONS

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

On way to produce the disclosed proteins, polypeptides, or peptides is to express the protein in a cell from an expression vector comprising nucleic acids encoding the proteins, polypeptides, or peptides, such as those disclosed herein.

Another method of producing the disclosed proteins, such as SEQ ID NOs:1, 2, 3, 4, 5, 6, 7, or 15, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides can be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

### C. DEFINITIONS

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents.

It must be noted that as used herein and in the appended claims, the singular forms “a,” “an,” and “the” include plural reference unless the context clearly dictates otherwise. Thus, for example, reference to “a peptide” includes a plurality of such peptides, reference to “the peptide” is a reference to one or more peptides and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Throughout the description and claims of this specification, the word “comprise” and variations of the word, such as “comprising” and “comprises,” means “including but not limited to,” and is not intended to exclude, for example, other additives, components, integers or steps.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### i. Methods

Ubiquitin chain assembly on Ubc13 is monitored based on the principle of TR-FRET. Bacteria-produced recombinant ubiquitin conjugating enzymes, His-hUbc13 and His-hUev1a, were used. Ubiquitination reaction mixture consisted of ubiquitin-activating enzyme (His-E1, 12.5 nM), hUbc13/hUev1a (His-E2, 250 nM each), terbium-labeled ubiquitin (10 nM), fluorescein-labeled ubiquitin (150 nM), and an ATP regenerating system (consisting of 1 mM ATP, 1.25 mM MgCl₂, 2.5 mM phosphocreatine, and 0.035 units/μL creatine phosphokinase). Ubiquitination reactions were set up in a standard black 384-well plate. Assay buffer was 50 mM Hepes pH 7.5/100 mM NaCl/0.005% Empigen BB detergent/0.1 mM DTT/1% DMSO. Stock solutions of ubiquitin, E1, E2, and ATP regenerating system were prepared in 50 mM Hepes pH 7.5 buffer. The procedure involved the following. Buffer components, E1, E2, terbium-labeled ubiquitin, and ATP-regenerating system (in the amounts mentioned above) were added into 384-well plate, mixed while adding each of the four components, and incubated at 37° C. or RT for 5 min. Following this, fluorescein-ubiquitin was added to the reaction mix and the plate incubated at 37° C. The plate was read at regular time intervals (1 hr, 3 hr, 5 hr etc.) in TR-FRET mode on a Molecular Devices instrument (Analyst®). Terbium readings were taken at 360/480 nm and fluorescein was read at 360/520 nm. A graphical analysis was generated by plotting the ratio of the intensities of the acceptor and donor fluorophores (Emission ratio 520/480 nm) for each set of reaction mixtures. Fold-increase in TR-FRET signal for each data set was determined with respect to control readings (either terbium-Ub+fluorescein-Ub or terbium-Ub+fluorescein-Ub+ATP regenerating system or reaction mix lacking E2). Data was represented as mean+/−SD. The assay was optimized for E2 concentration, salt concentration, DTT, and temperature.

### ii. Results

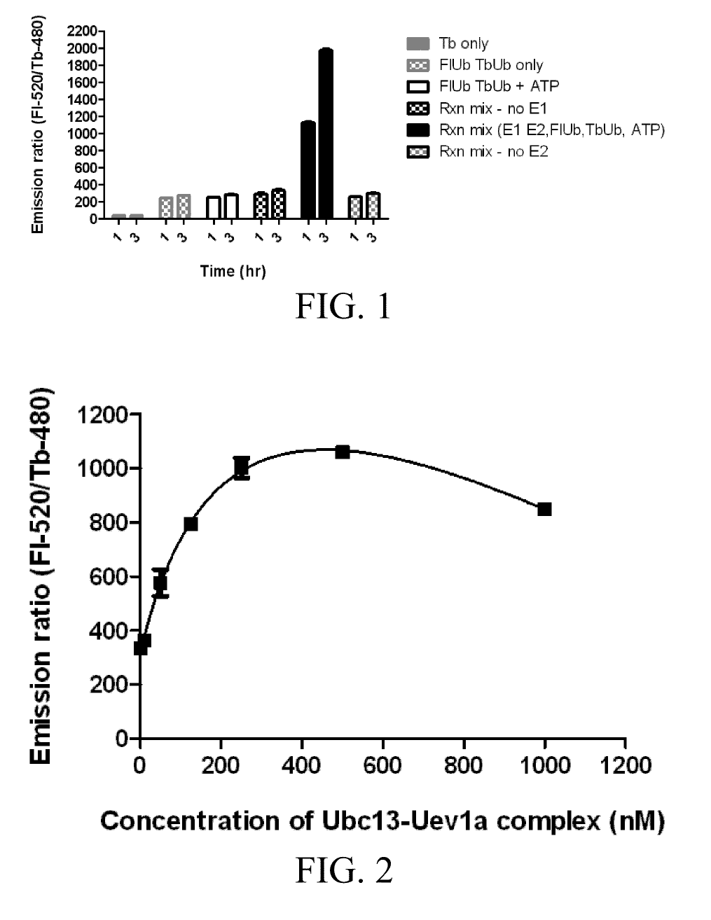

Ubiquitin chain assembly mediated by Ubc13-Uev1a complex was monitored based on TR-FRET assay methodology as described above. Signal:Noise (S/N) observed was around 4-fold (1 hr time point) to 8-fold (3 hr time point). Time-dependent TR-FRET responses for ubiquitination and control reaction systems are shown in FIG. 1.

To determine the optimal concentration of Ubc13-Uev1a, ubiquitination reaction was performed as described and the assay was done under varying concentrations of Ubc13-Uev1a complex (ranging from 0-1000 nM). Data processing was done in the same manner and non-linear regression analysis of the data was done using PRISM v. 5.0 for observation of the enzymatic activity of the complex. The optimal concentration of Ubc13-Uev1a complex was around 250 nM (FIG. 2).

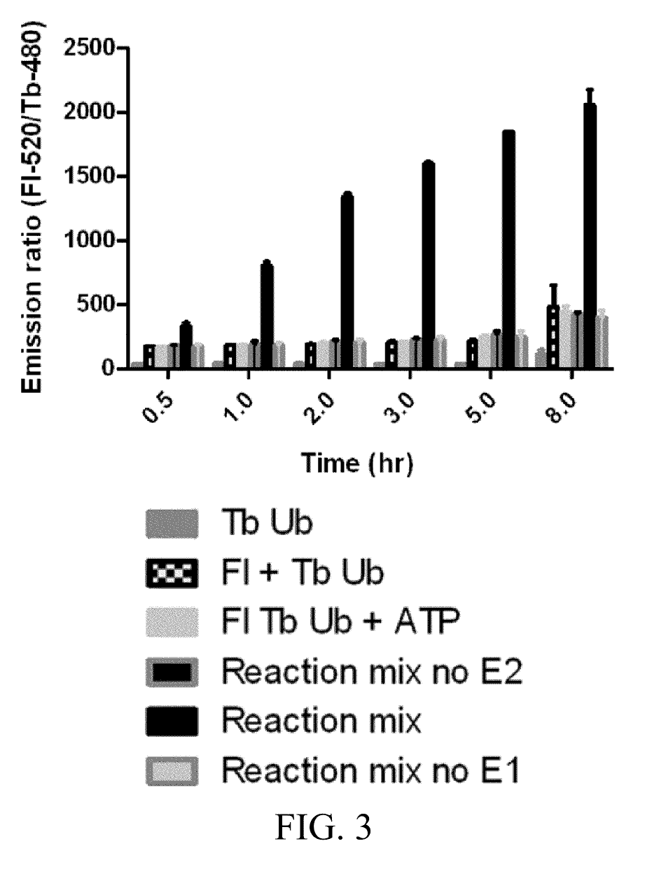

To determine the optimal concentrations of the assay, ubiquitination reaction was performed as described and taken at regular time points. Data processing was done in the same manner as described using PRISM v. 5.0. Data at optimal concentration of E2 (250 nM) is shown in FIG. 3. The optimal time point seemed to be between 1 and 3 hr.

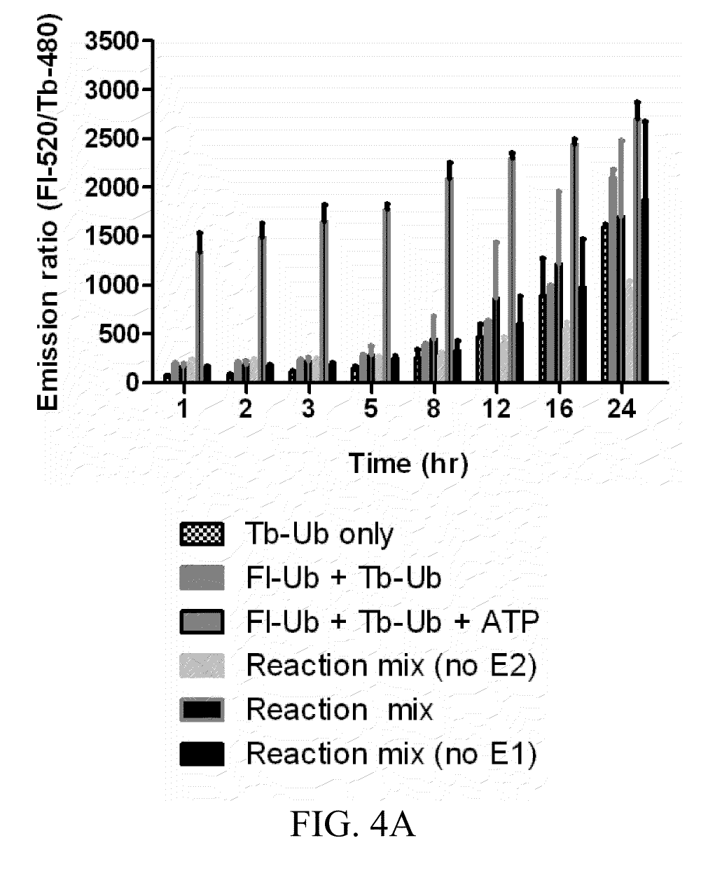

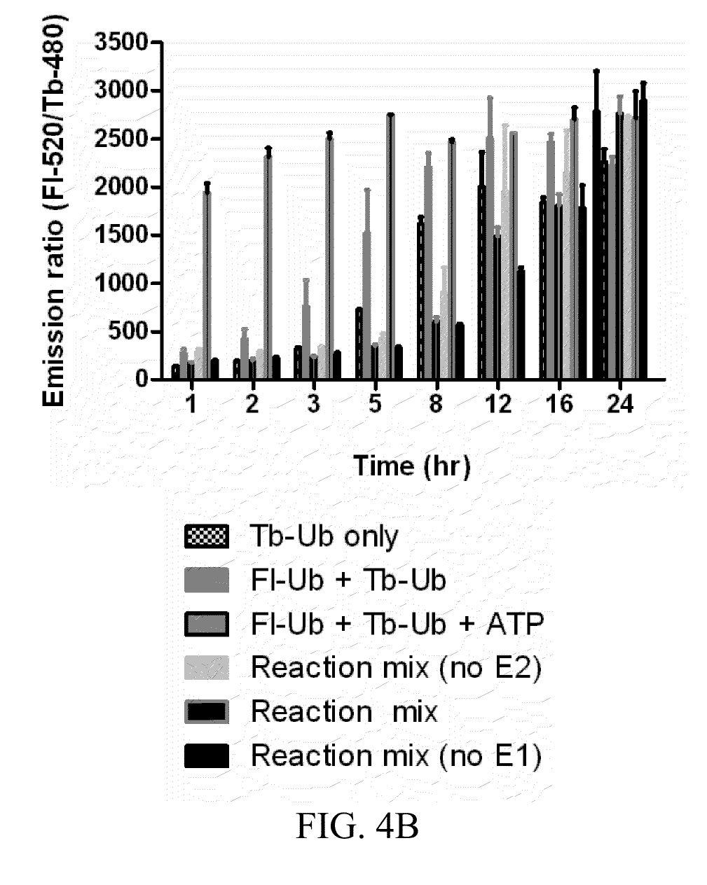

Ubiquitination reactions were performed as described in the method section at incubation temperatures of 37° C. and RT. Optimal reaction temperature was determined based on TR-FRET signal and on the stability of TR-FRET signal over time at these two incubation conditions. Data processing was done in the same manner as described using PRISM v. 5.0. and represented in FIG. 4. TR-FRET signal was stable over time when incubations were performed at RT.

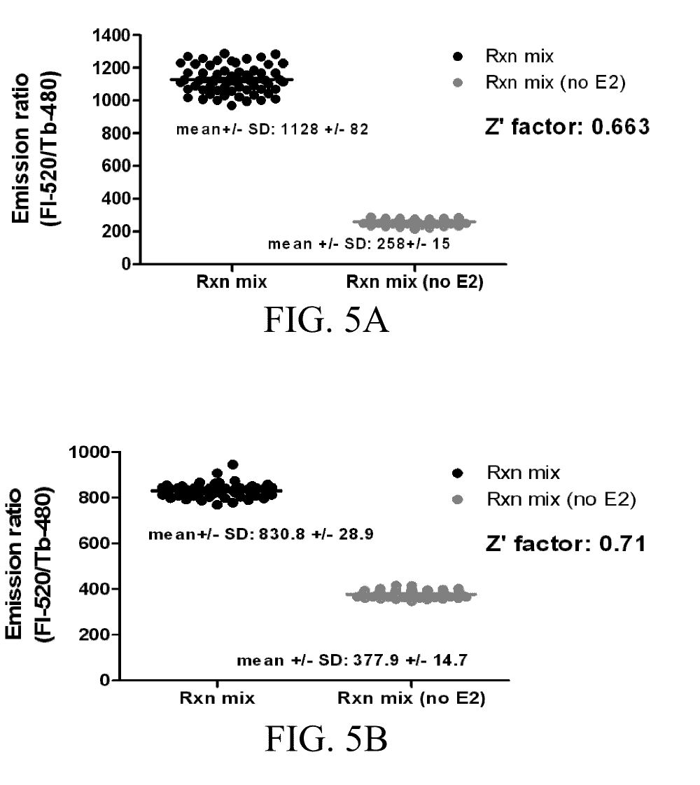

The ‘screening window coefficient’ called Z′ factor was determined for assessing the reliability and reproducibility of the assay with respect to signal to noise ratio. This was done by comparing the dynamic range of the assay to data variability. In the TR-FRET assay system, the Z′ factor was calculated for the complete reaction mixture and this factor was compared to a reaction lacking Ubc13/Uev1a in the mixture. The equation used for determining the Z′ factor was

$${Z}^{′} ≡ 1 - ( \frac{3 {σ}_{1} + 3 {σ}_{2 }}{{μ}_{2} - {μ}_{1}} )$$

where σ₁ and σ₂ are the standard deviations of the low and high controls, respectively and μ₁ and μ₂ are the means of the low and high controls. The mean values were obtained for positive (complete reaction mixture) and negative (reaction mixture lacking Ubc13/Uev1a) controls present in the assay. Data from Z′ factor determination experiments done at 37° C. and RT are shown in FIG. 5. Z′˜0.7.

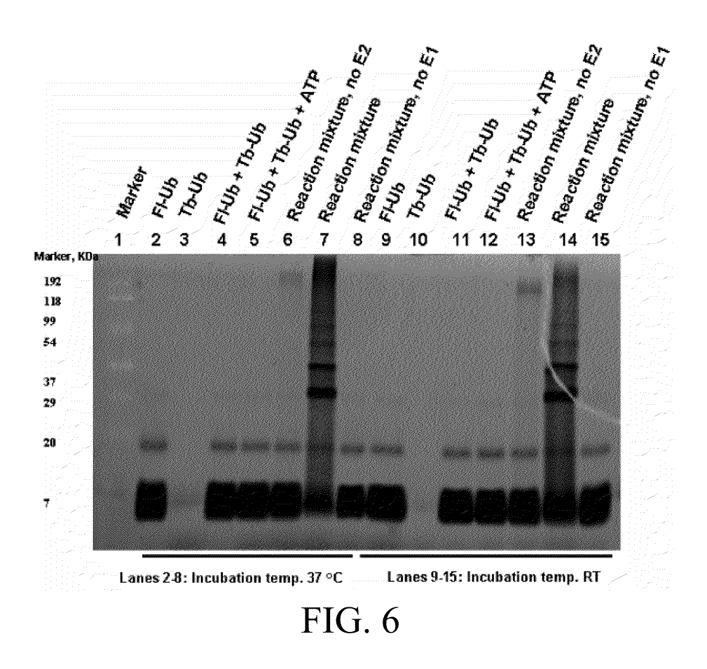

Aliquots from reactions performed based on TR FRET methodology were confirmed for ubiquitination based on 15% SDS-PAGE analysis. Fluorescein-ubiquitin chain assembly under different reaction conditions was detected by scanning the gel using Fluorimager. Data is shown in FIG. 6.

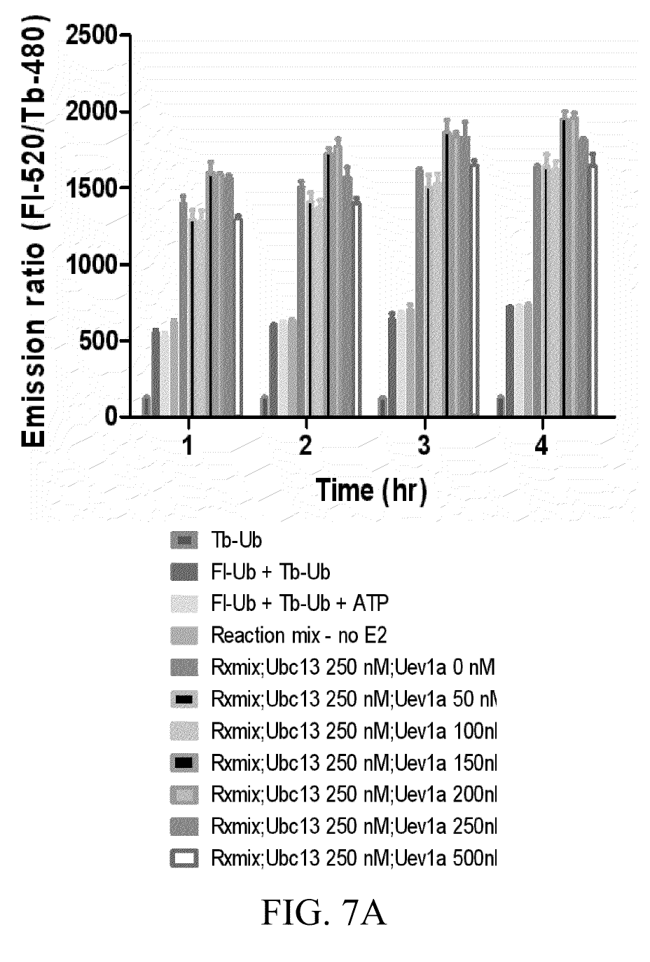

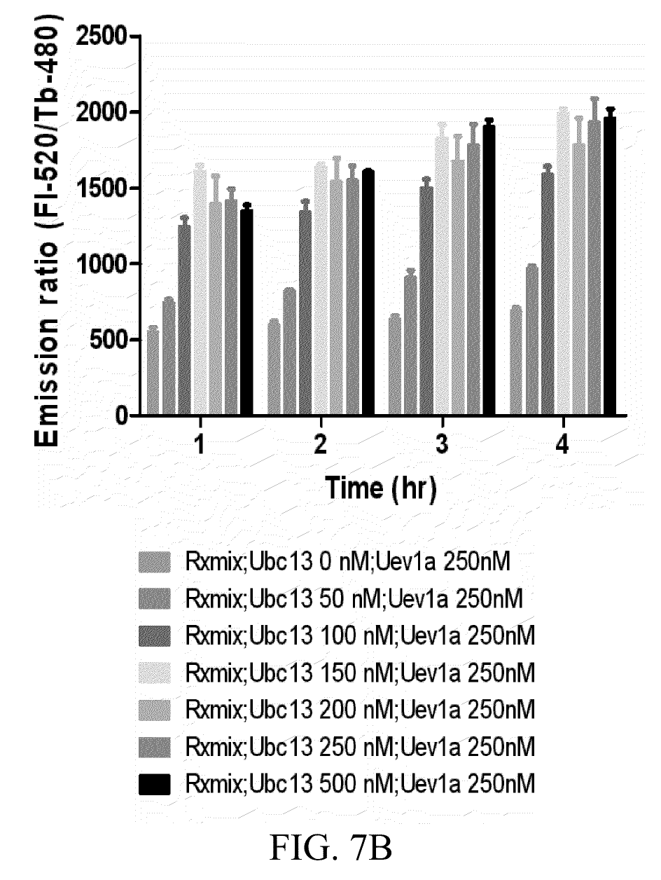

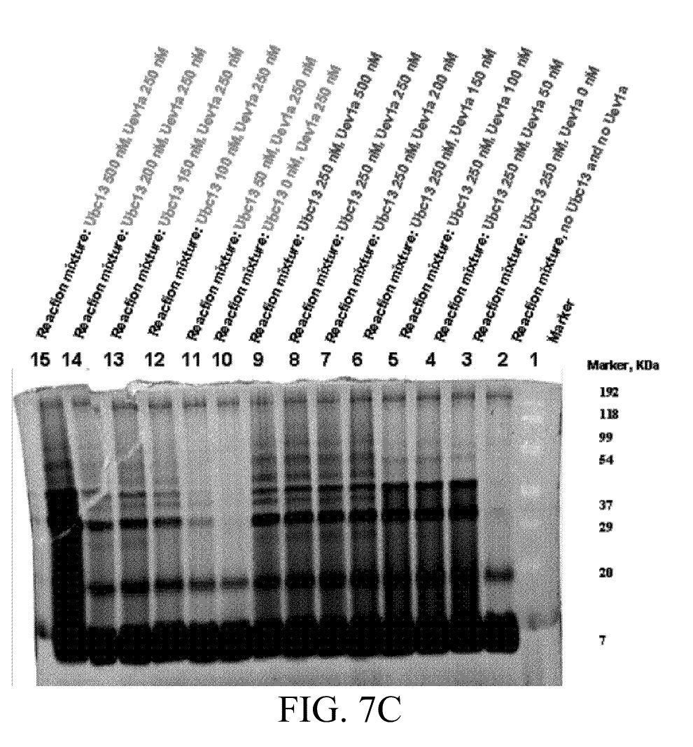

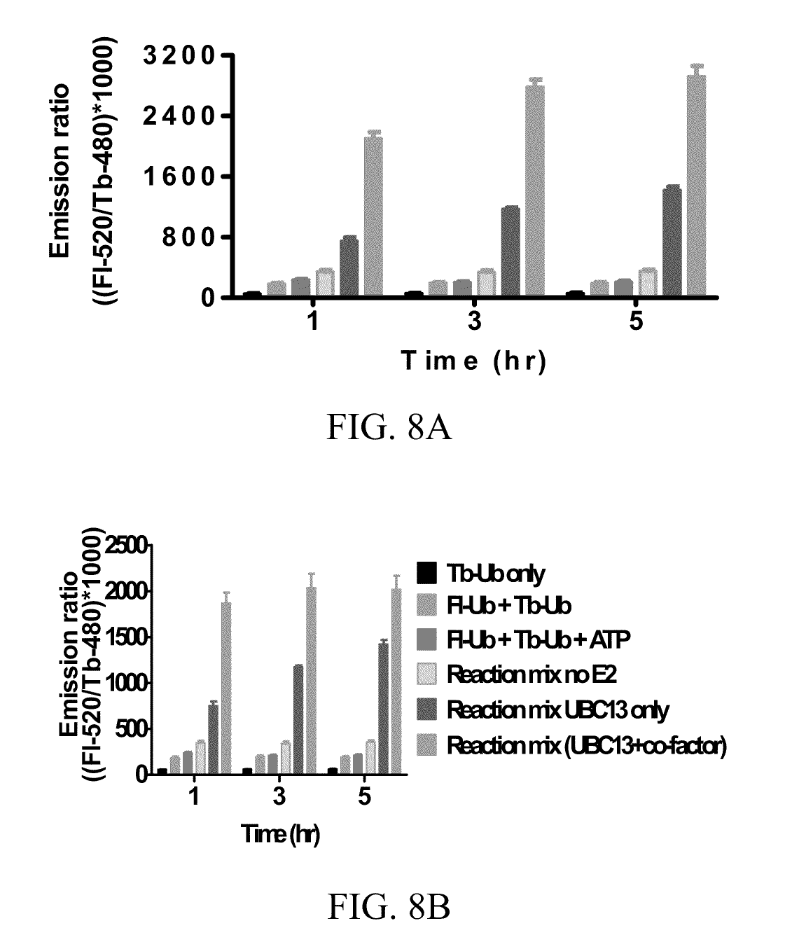

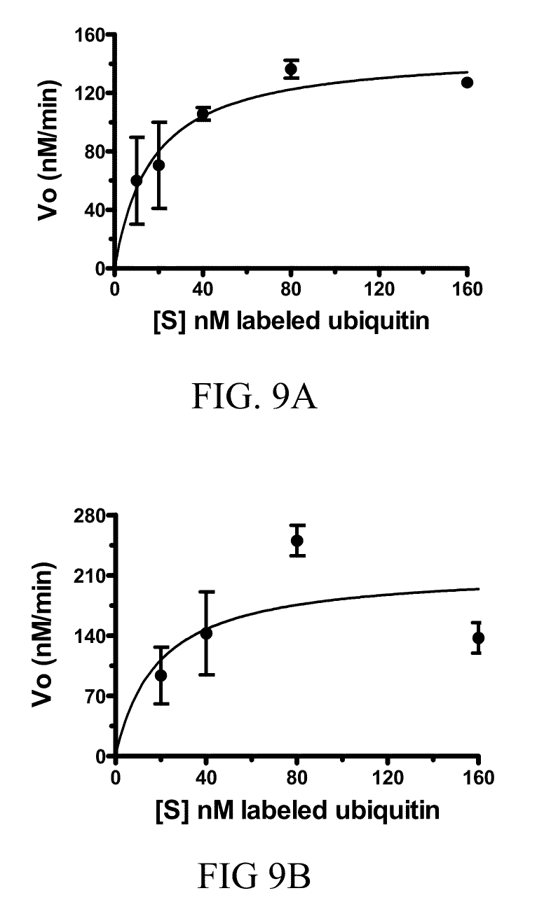

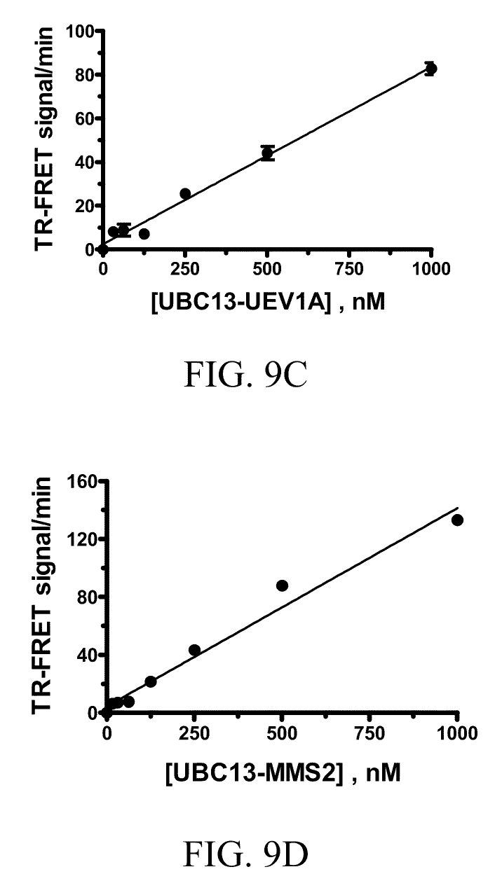

The polyubiquitin chain formation on Uev1a was monitored by TR-FRET methodology using either varying concentrations of Ubc13 or Uev1a and fixing the concentration of one of the two heterodimeric partners at a time. Diubiquitination of Ubc13 was followed by ubiquitin transfer to and subsequent polyubiquitination of Uev1a. Aliquots from TR-FRET were analyzed by 15% SDS-PAGE analysis. Data is shown in FIGS. 7a-c.

### E. SEQUENCES

1. (Human Ubiquitin-Accession P02248)

SEQ ID NO: 1

mqifvktltg ktitleveps dtienvkaki qdkegippdq
qrlifagkql edgrtlsdyn iqkestlhlv lrlrgg
2. (Human Ubc13)

SEQ ID NO: 2

maglprriik etqrllaepv pgikaepdes naryfhvvia
gpqdspfegg tfklelflpe eypmaapkvr fmtkiyhpnv
dklgricldi lkdkwspalq irtvllsiqa llsapnpddp
landvaeqwk tneaqaieta rawtrlyamn ni
3. (Human Uev1a)

SEQ ID NO: 3

mpgevqasyl ksqsklsdeg rleprkfhck gvkvprnfrl
leeleegqkg vgdgtvswgl eddedmtltr wtgmiigppr
tiyenriysl kiecgpkype appfvrfvtk inmngvnssn
gvvdpraisv lakwqnsysi kvvlqelrrl mmskenmklp
qppegqcysn
4. (Human E1)

SEQ ID NO: 4

msssplskkr rvsgpdpkpg sncspaqsvl sevpsvptng
makngseadi deglysrqly vlgheamkrl qtssvlvsgl
rglgveiakn iilggvkavt lhdqgtaqwa dlssqfylre
edigknraev sqprlaelns yvpvtaytgp lvedflsgfq
vvvltntple dqlrvgefch nrgiklvvad trglfgqlfc
dfgeemiltd sngeqplsam vsmvtkdnpg vvtcldearh
gfesgdfvsf sevqgmveln gnqpmeikvl gpytfsicdt
snfsdyirgg ivsqvkvpkk isfkslvasl aepdfvvtdf
akfsrpaqlh igfqalhqfc aqhgrpprpr needaaelva
laqavnaral pavqqnnlde dlirklayva agdlapinaf
igglaaqevm kacsgkfmpi mqwlyfdale clpedkevlt
edkclqrqnr ydgqvavfgs dlqeklgkqk yflvgagaig
cellknfami glgcgeggei ivtdmdtiek snlnrqflfr
pwdvtklksd taaaavrqmn phirvtshqn rvgpdteriy
dddffqnldg vanaldnvda rmymdrrcvy yrkpllesgt
lgtkgnvqvv ipfltesyss sqdppeksip ictlknfpna
iehtlqward efeglfkqpa envnqyltdp kfvertlrla
gtqplevlea vqrslvlqrp qtwadcvtwa chhwhtqysn
nirqllhnfp pdqltssgap fwsgpkrcph pltfdvnnpl
hldyvmaaan lfaqtygltg sqdraavatf lqsvqvpeft
pksgvkihvs dqelqsanas vddsrleelk atlpspdklp
gfkmypidfe kdddsnfhmd fivaasnlra enydipsadr
hkskliagki ipaiatttaa vvglvclely kvvqghrqld
sykngflnla lpffgfsepl aaprhqyynq ewtlwdrfev
qglqpngeem tlkqfldyfk tehkleitml sqgvsmlysf
fmpaaklker ldqpmteivs rvskrklgrh vralvlelcc
ndesgedvev pyvrytir
5. (UBA2)

SEQ ID NO: 5

malsrglpre laeavaggrv lvvgaggigc ellknlvltg
fshidlidld tidvsnlnrq flfqkkhvgr skaqvakesv
lqfypkaniv ayhdsimnpd ynveffrqfi lvmnaldnra
arnhvnrmcl aadvpliesg tagylgqvtt ikkgvtecye
chpkptqrtf pgctirntps epihcivwak ylfnqlfgee
dadqevspdr adpeaawept eaeararasn edgdikrist
kewakstgyd pvklftklfk ddirylltmd klwrkrkppv
pldwaevqsq geetnasdqq nepqlglkdq qvldvksyar
lfsksietlr vhlaekgdga eliwdkddps amdfvtsaan
lrmhifsmnm ksrfdiksma gniipaiatt naviaglivl
eglkilsgki dqcrtiflnk qpnprkkllv pcaldppnpn
cyvcaskpev tvrlnvhkvt vltlqdkivk ekfamvapdv
qiedgkgtil isseegetea nnhkklsefg irngsrlqad
dflqdytlli nilhsedlgk dvefevvgda pekvgpkqae
daaksitngs ddgaqpstst aqeqddvliv dsdeedssnn
advseeersr krkldekenl sakrsrieqk eelddviald
6. (Human Uev1c)

SEQ ID NO: 6

mkedlnlenf taktiyenri yslkiecgpk ypeappfvrf
vtkinmngvn ssngvvdpra isvlakwqns ysikvvlqel
rrlmmskenm klpqppegqc ysn
7. (Human Uev1d)

SEQ ID NO: 7

maattgsgvk vprnfrllee leegqkgvgd gtvswgledd
edmtltrwtg miigpprtiy enriyslkie cgpkypeapp
fvrfvtkinm ngvnssngvv dpraisvlak wqnsysikvv
lqelrrlmms kenmklpqpp egqcysn
8. (Human Uev1a variant 1-Accession NM_021988)

SEQ ID NO: 8

cccgcctaac ctcttcctgc gatgagctcg gcacgggaat
tattattgtc aattttactt gcaagaagtt tcctacaaga
gccaaggaat ccatgcgagt aaacatttac gggcaccata
gataaaaggc ttgtgtttta atcctcatcc tctccacctg
ttagctctga gtctcagttt tctcatctct aaaaatgggg
atattcacag gagttgctgc atcgagttgt gaggattaaa
agttggatgt aacggcttgg taattatgag ctcttctagt
gtcccttcct cttccctgtg cccaaggggt tttaggaaag
cattttatct ccacagcaat cctatgaggt tgatactact
atcctcatag aaggggaaac tgatgccagg agaggttcaa
gcgtcttacc tgaagtcaca aagcaaactg agtgatgaag
gaagacttga acctagaaaa tttcactgca aaggagtaaa
agtccctcgc aatttccgac tgttggaaga actcgaagaa
ggccagaaag gagtaggaga tggcacagtt agctggggtc
tagaagatga cgaagacatg acacttacaa gatggacagg
gatgataatt gggcctccaa gaacaattta tgaaaaccga
atatacagcc ttaaaataga atgtggacct aaatacccag
aagcaccccc ctttgtaaga tttgtaacaa aaattaatat
gaatggagta aatagttcta atggagtggt ggacccaaga
gccatatcag tgctagcaaa atggcagaat tcatatagca
tcaaagttgt cctgcaagag cttcggcgcc taatgatgtc
taaagaaaat atgaaactcc ctcagccgcc cgaaggacag
tgttacagca attaatcaaa aagaaaaacc acaggccctt
ccccttcccc ccaattcgat ttaatcagtc ttcattttcc
acagtagtaa attttctaga tacgtcttgt agacctcaaa
gtaccggaaa ggaagctccc attcaaagga aatttatctt
aagatactgt aaatgatact aattttttgt ccatttgaaa
tatataagtt gtgctataac aaatcatcct gtcaagtgta
accactgtcc acgtagttga acttctggga tcaagaaagt
ctatttaaat tgattcccat cataactggt ggggcacatc
taactcaact gtgaaaagac acatcacaca atcaccttgc
tgctgattac acggcctggg gtctctgcct tctcccctta
ccctcccgcc tcccaccctc cctgcaacaa cagccctcta
gcctgggggg cttgttagag tagatgtgaa ggtttcaggt
cgcagcctgt gggactactg ctaggtgtgt ggggtgtttc
gcctgcaccc ctggtttctt taagtcttaa gtgatgcccc
ttccaaacca tcatcctgtc cccacgctcc tccactcccg
cccttggccg aagcatagat tgtaacccct ccactcccct
ctgagattgg ccttcggtga ggaattcagg gctttcccca
tatcttctct cccccacctt tatcgagggg tgctgctttt
tctccctcct cctcaagttc ctttttgcac cgtcaccacc
caacaccttc catgacactt ccttgctttg gccagaagcc
atcaggtaag gttggaaaga gcctctgacc tcccttgttt
agttttggaa ccatactcac tcactctcca ccagcctggg
aaatgaatat tgggtcctca gccctgccac cctctgctgt
catcagctga tgcattgttt ttagctcagg ttttgataag
gtgaaaagaa tagtcaccag ggttactcag acctgccagc
tctcggagtc cttggtggtt gaacttggag aaagaccgca
tgaagatact tgtaagcaca catgatccct ctgaattgtt
ttactttcct gtaactgctt ttgcttttaa aaattgaaga
agttttaaac agggctttca tttggtcatc cttgcaatcc
attggggtct agtttggaat ctgacaactg gaacaaaaag
aaccttgaat ccggtgcatg ccttggtttt ggtgctgctg
ctgcttccca agatcctcag cagggattaa gaaggaaccc
ggtgtgcaca gcagatcccc gaaattggtg ggcttgacct
cctggcaaat tgctgcgtct ttccacttgc tgttcaggac
cactaaatgc tgaaatgtgg atgcataccg aaataaaagc
aattcattgt gtactaaagg tttttttttt ttttttaatt
tagtatttgt gtaaaaccac cttttgaagc agcaactatc
aagtctgaaa agcaattgat gtttccatta atctttttct
ggggggaaaa ccttagttct aaggatttaa catcctgtaa
gtgaagttta acataacagt attccataag cagccttttt
attgtcagac cattgcctga ttttaatata ataaaaaaaa
agtgtgcgtt aatatttaa
9. (Human Uev1a variant 2-Accession NP_954595)

SEQ ID NO: 9

cccgcctaac ctcttcctgc gatgagctcg gcacgggaat
tattattgtc aattttactt gcaagaagtt tcctacaaga
gccaaggaat ccatgcgagt aaacatttac gggcaccata
gataaaaggc ttgtgtttta atcctcatcc tctccacctg
ttagctctga gtctcagttt tctcatctct aaaaatgggg
atattcacag gagttgctgc atcgagttgt gaggattaaa
agttggatgt aacggcttgc aatcctatga ggttgatact
actatcctca tagaagggga aactgatgcc aggagaggtt
caagcgtctt acctgaagtc acaaagcaaa ctgagtgatg
aaggaagact tgaacctaga aaatttcact gcaaaggagt
aaaagtccct cgcaatttcc gactgttgga agaactcgaa
gaaggccaga aaggagtagg agatggcaca gttagctggg
gtctagaaga tgacgaagac atgacactta caagatggac
agggatgata attgggcctc caagaacaat ttatgaaaac
cgaatataca gccttaaaat agaatgtgga cctaaatacc
cagaagcacc cccctttgta agatttgtaa caaaaattaa
tatgaatgga gtaaatagtt ctaatggagt ggtggaccca
agagccatat cagtgctagc aaaatggcag aattcatata
gcatcaaagt tgtcctgcaa gagcttcggc gcctaatgat
gtctaaagaa aatatgaaac tccctcagcc gcccgaagga
cagtgttaca gcaattaatc aaaaagaaaa accacaggcc
cttccccttc cccccaattc gatttaatca gtcttcattt
tccacagtag taaattttct agatacgtct tgtagacctc
aaagtaccgg aaaggaagct cccattcaaa ggaaatttat
cttaagatac tgtaaatgat actaattttt tgtccatttg
aaatatataa gttgtgctat aacaaatcat cctgtcaagt
gtaaccactg tccacgtagt tgaacttctg ggatcaagaa
agtctattta aattgattcc catcataact ggtggggcac
atctaactca actgtgaaaa gacacatcac acaatcacct
tgctgctgat tacacggcct ggggtctctg ccttctcccc
ttaccctccc gcctcccacc ctccctgcaa caacagccct
ctagcctggg gggcttgtta gagtagatgt gaaggtttca
ggtcgcagcc tgtgggacta ctgctaggtg tgtggggtgt
ttcgcctgca cccctggttt ctttaagtct taagtgatgc
cccttccaaa ccatcatcct gtccccacgc tcctccactc
ccgcccttgg ccgaagcata gattgtaacc cctccactcc
cctctgagat tggccttcgg tgaggaattc agggctttcc
ccatatcttc tctcccccac ctttatcgag gggtgctgct
ttttctccct cctcctcaag ttcctttttg caccgtcacc
acccaacacc ttccatgaca cttccttgct ttggccagaa
gccatcaggt aaggttggaa agagcctctg acctcccttg
tttagttttg gaaccatact cactcactct ccaccagcct
gggaaatgaa tattgggtcc tcagccctgc caccctctgc
tgtcatcagc tgatgcattg tttttagctc aggttttgat
aaggtgaaaa gaatagtcac cagggttact cagacctgcc
agctctcgga gtccttggtg gttgaacttg gagaaagacc
gcatgaagat acttgtaagc acacatgatc cctctgaatt
gttttacttt cctgtaactg cttttgcttt taaaaattga
agaagtttta aacagggctt tcatttggtc atccttgcaa
tccattgggg tctagtttgg aatctgacaa ctggaacaaa
aagaaccttg aatccggtgc atgccttggt tttggtgctg
ctgctgcttc ccaagatcct cagcagggat taagaaggaa
cccggtgtgc acagcagatc cccgaaattg gtgggcttga
cctcctggca aattgctgcg tctttccact tgctgttcag
gaccactaaa tgctgaaatg tggatgcata ccgaaataaa
agcaattcat tgtgtactaa aggttttttt ttttttttta
atttagtatt tgtgtaaaac caccttttga agcagcaact
atcaagtctg aaaagcaatt gatgtttcca ttaatctttt
tctgggggga aaaccttagt tctaaggatt taacatcctg
taagtgaagt ttaacataac agtattccat aagcagcctt
tttattgtca gaccattgcc tgattttaat ataataaaaa
aaaagtgtgc gttaatattt aaaaaaaaaa aaaaaaa
10. (Human Uev1b-Accession NM_022442)

SEQ ID NO: 10

gcctaacctc ttcctgcgat gagctcggca cgggaattat
tattgtcaat tttacttgca agaagtttcc tacaagagcc
aaggaatcca tgcgagtaaa catttacggg caccatagat
aaaagcaatc ctatgaggtt gatactacta tcctcataga
aggggaaact gatgccagga gaggttcaag cgtcttacct
gaagtcacaa agcaaactga gtgatgaagg aagacttgaa
cctagaaaat ttcactgcaa agacaattta tgaaaaccga
atatacagcc ttaaaataga atgtggacct aaatacccag
aagcaccccc ctttgtaaga tttgtaacaa aaattaatat
gaatggagta aatagttcta atggagtggt ggacccaaga
gccatatcag tgctagcaaa atggcagaat tcatatagca
tcaaagttgt cctgcaagag cttcggcgcc taatgatgtc
taaagaaaat atgaaactcc ctcagccgcc cgaaggacag
tgttacagca attaatcaaa aagaaaaacc acaggccctt
ccccttcccc ccaattcgat ttaatcagtc ttcattttcc
acagtagtaa attttctaga tacgtcttgt agacctcaaa
gtaccggaaa ggaagctccc attcaaagga aatttatctt
aagatactgt aaatgatact aattttttgt ccatttgaaa
tatataagtt gtgctataac aaatcatcct gtcaagtgta
accactgtcc acgtagttga acttctggga tcaagaaagt
ctatttaaat tgattcccat cataactggt ggggcacatc
taactcaact gtgaaaagac acatcacaca atcaccttgc
tgctgattac acggcctggg gtctctgcct tctcccctta
ccctcccgcc tcccaccctc cctgcaacaa cagccctcta
gcctgggggg cttgttagag tagatgtgaa ggtttcaggt
cgcagcctgt gggactactg ctaggtgtgt ggggtgtttc
gcctgcaccc ctggtttctt taagtcttaa gtgatgcccc
ttccaaacca tcatcctgtc cccacgctcc tccactcccg
cccttggccg aagcatagat tgtaacccct ccactcccct
ctgagattgg ccttcggtga ggaattcagg gctttcccca
tatcttctct cccccacctt tatcgagggg tgctgctttt
tctccctcct cctcaagttc ctttttgcac cgtcaccacc
caacaccttc catgacactt ccttgctttg gccagaagcc
atcaggtaag gttggaaaga gcctctgacc tcccttgttt
agttttggaa ccatactcac tcactctcca ccagcctggg
aaatgaatat tgggtcctca gccctgccac cctctgctgt
catcagctga tgcattgttt ttagctcagg ttttgataag
gtgaaaagaa tagtcaccag ggttactcag acctgccagc
tctcggagtc cttggtggtt gaacttggag aaagaccgca
tgaagatact tgtaagcaca catgatccct ctgaattgtt
ttactttcct gtaactgctt ttgcttttaa aaattgaaga
agttttaaac agggctttca tttggtcatc cttgcaatcc
attggggtct agtttggaat ctgacaactg gaacaaaaag
aaccttgaat ccggtgcatg ccttggtttt ggtgctgctg
ctgcttccca agatcctcag cagggattaa gaaggaaccc
ggtgtgcaca gcagatcccc gaaattggtg ggcttgacct
cctggcaaat tgctgcgtct ttccacttgc tgttcaggac
cactaaatgc tgaaatgtgg atgcataccg aaataaaagc
aattcattgt gtactaaagg tttttttttt ttttttaatt
tagtatttgt gtaaaaccac cttttgaagc agcaactatc
aagtctgaaa agcaattgat gtttccatta atctttttct
ggggggaaaa ccttagttct aaggatttaa catcctgtaa
gtgaagttta acataacagt attccataag cagccttttt
attgtcagac cattgcctga ttttaatata ataaaaaaaa
agtgtgcgtt aatatttaa
11. (Human Uev1d-Accession NM_001032288)

SEQ ID NO: 11

gggggggtga agaaggggcc ggccttcaag caagagcgac
gcaagatggc agccaccacg ggctcgggag taaaagtccc
tcgcaatttc cgactgttgg aagaactcga agaaggccag
aaaggagtag gagatggcac agttagctgg ggtctagaag
atgacgaaga catgacactt acaagatgga cagggatgat
aattgggcct ccaagaacaa tttatgaaaa ccgaatatac
agccttaaaa tagaatgtgg acctaaatac ccagaagcac
ccccctttgt aagatttgta acaaaaatta atatgaatgg
agtaaatagt tctaatggag tggtggaccc aagagccata
tcagtgctag caaaatggca gaattcatat agcatcaaag
ttgtcctgca agagcttcgg cgcctaatga tgtctaaaga
aaatatgaaa ctccctcagc cgcccgaagg acagtgttac
agcaattaat caaaaagaaa aaccacaggc ccttcccctt
ccccccaatt cgatttaatc agtcttcatt ttccacagta
gtaaattttc tagatacgtc ttgtagacct caaagtaccg
gaaaggaagc tcccattcaa aggaaattta tcttaagata
ctgtaaatga tactaatttt ttgtccattt gaaatatata
agttgtgcta taacaaatca tcctgtcaag tgtaaccact
gtccacgtag ttgaacttct gggatcaaga aagtctattt
aaattgattc ccatcataac tggtggggca catctaactc
aactgtgaaa agacacatca cacaatcacc ttgctgctga
ttacacggcc tggggtctct gccttctccc cttaccctcc
cgcctcccac cctccctgca acaacagccc tctagcctgg
ggggcttgtt agagtagatg tgaaggtttc aggtcgcagc
ctgtgggact actgctaggt gtgtggggtg tttcgcctgc
acccctggtt tctttaagtc ttaagtgatg ccccttccaa
accatcatcc tgtccccacg ctcctccact cccgcccttg
gccgaagcat agattgtaac ccctccactc ccctctgaga
ttggccttcg gtgaggaatt cagggctttc cccatatctt
ctctccccca cctttatcga ggggtgctgc tttttctccc
tcctcctcaa gttccttttt gcaccgtcac cacccaacac
cttccatgac acttccttgc tttggccaga agccatcagg
taaggttgga aagagcctct gacctccctt gtttagtttt
ggaaccatac tcactcactc tccaccagcc tgggaaatga
atattgggtc ctcagccctg ccaccctctg ctgtcatcag
ctgatgcatt gtttttagct caggttttga taaggtgaaa
agaatagtca ccagggttac tcagacctgc cagctctcgg
agtccttggt ggttgaactt ggagaaagac cgcatgaaga
tacttgtaag cacacatgat ccctctgaat tgttttactt
tcctgtaact gcttttgctt ttaaaaattg aagaagtttt
aaacagggct ttcatttggt catccttgca atccattggg
gtctagtttg gaatctgaca actggaacaa aaagaacctt
gaatccggtg catgccttgg ttttggtgct gctgctgctt
cccaagatcc tcagcaggga ttaagaagga acccggtgtg
cacagcagat ccccgaaatt ggtgggcttg acctcctggc
aaattgctgc gtctttccac ttgctgttca ggaccactaa
atgctgaaat gtggatgcat accgaaataa aagcaattca
ttgtgtacta aaggtttttt tttttttttt aatttagtat
ttgtgtaaaa ccaccttttg aagcagcaac tatcaagtct
gaaaagcaat tgatgtttcc attaatcttt ttctgggggg
aaaaccttag ttctaaggat ttaacatcct gtaagtgaag
tttaacataa cagtattcca taagcagcct ttttattgtc
agaccattgc ctgattttaa tataataaaa aaaaagtgtg
cgttaatatt taaaaaaa
12. (Human E1 (UBA1)-Accession NM_153280)

SEQ ID NO: 12

tcccagaccc ggggctctcc aaggccccgc gcttccgagc
tccgcgcaaa ctctggcttc tcttgtacga cagaggtggt
ttgctcttcc gttgccccgt ggcttcagct catctttggc
aggaaggcga ggcttccgcc cggcacaggg gatgtccagc
tcgccgctgt ccaagaaacg tcgcgtgtcc gggcctgatc
caaagccggg ttctaactgc tcccctgccc agtccgtgtt
gtccgaagtg ccctcggtgc caaccaacgg aatggccaag
aacggcagtg aagcagacat agacgagggc ctttactccc
ggcagctgta tgtgttgggc catgaggcaa tgaagcggct
ccagacatcc agtgtcctgg tatcaggcct gcggggcctg
ggcgtggaga tcgctaagaa catcatcctt ggtggggtca
aggctgttac cctacatgac cagggcactg cccagtgggc
tgatctttcc tcccagttct acctgcggga ggaggacatc
ggtaaaaacc gggccgaggt atcacagccc cgcctcgctg
agctcaacag ctatgtgcct gtcactgcct acactggacc
cctcgttgag gacttcctta gtggtttcca ggtggtggtg
ctcaccaaca cccccctgga ggaccagctg cgagtgggtg
agttctgtca caaccgtggc atcaagctgg tggtggcaga
cacgcggggc ctgtttgggc agctcttctg tgactttgga
gaggaaatga tcctcacaga ttccaatggg gagcagccac
tcagtgctat ggtttctatg gttaccaagg acaaccccgg
tgtggttacc tgcctggatg aggcccgaca cgggtttgag
agcggggact ttgtctcctt ttcagaagta cagggcatgg
ttgaactcaa cggaaatcag cccatggaga tcaaagtcct
gggtccttat acctttagca tctgtgacac ctccaacttc
tccgactaca tccgtggagg catcgtcagt caggtcaaag
tacctaagaa gattagcttt aaatccttgg tggcctcact
ggcagaacct gactttgtgg tgacggactt cgccaagttt
tctcgccctg cccagctgca cattggcttc caggccctgc
accagttctg tgctcagcat ggccggccac ctcggccccg
caatgaggag gatgcagcag aactggtagc cttagcacag
gctgtgaatg ctcgagccct gccagcagtg cagcaaaata
acctggacga ggacctcatc cggaagctgg catatgtggc
tgctggggat ctggcaccca taaacgcctt cattgggggc
ctggctgccc aggaagtcat gaaggcctgc tccgggaagt
tcatgcccat catgcagtgg ctatactttg atgcccttga
gtgtctccct gaggacaaag aggtcctcac agaggacaag
tgcctccagc gccagaaccg ttatgacggg caagtggctg
tgtttggctc agacctgcaa gagaagctgg gcaagcagaa
gtatttcctg gtgggtgcgg gggccattgg ctgtgagctg
ctcaagaact ttgccatgat tgggctgggc tgcggggagg
gtggagaaat catcgttaca gacatggaca ccattgagaa
gtcaaatctg aatcgacagt ttcttttccg gccctgggat
gtcacgaagt taaagtctga cacggctgct gcagctgtgc
gccaaatgaa tccacatatc cgggtgacaa gccaccagaa
ccgtgtgggt cctgacacgg agcgcatcta tgatgacgat
tttttccaaa acctagatgg cgtggccaat gccctggaca
acgtggatgc ccgcatgtac atggaccgcc gctgtgtcta
ctaccggaag ccactgctgg agtcaggcac actgggcacc
aaaggcaatg tgcaggtggt gatccccttc ctgacagagt
cgtacagttc cagccaggac ccacctgaga agtccatccc
catctgtacc ctgaagaact tccctaatgc catcgagcac
accctgcagt gggctcggga tgagtttgaa ggcctcttca
agcagccagc agaaaatgtc aaccagtacc tcacagaccc
caagtttgtg gagcgaacac tgcggctggc aggcactcag
cccttggagg tgctggaggc tgtgcagcgc agcctggtgc
tgcagcgacc acagacctgg gctgactgcg tgacctgggc
ctgccaccac tggcacaccc agtactcgaa caacatccgg
cagctgctgc acaacttccc tcctgaccag ctcacaagct
caggagcgcc gttctggtct gggcccaaac gctgtccaca
cccgctcacc tttgatgtca acaatcccct gcatctggac
tatgtgatgg ctgctgccaa cctgtttgcc cagacctacg
ggctgacagg ctctcaggac cgagctgctg tggccacatt
cctgcagtct gtgcaggtcc ccgaattcac ccccaagtct
ggcgtcaaga tccatgtttc tgaccaggag ctgcagagcg
ccaatgcctc tgttgatgac agtcgtctag aggagctcaa
agccactctg cccagcccag acaagctccc tggattcaag
atgtacccca ttgactttga gaaggatgat gacagcaact
ttcatatgga tttcatcgtg gctgcatcca acctccgggc
agaaaactat gacattcctt ctgcagaccg gcacaagagc
aagctgattg cagggaagat catcccagcc attgccacga
ccacagcagc cgtggttggc cttgtgtgtc tggagctgta
caaggttgtg caggggcacc gacagcttga ctcctacaag
aatggtttcc tcaacttggc cctgcctttc tttggtttct
ctgaacccct tgccgcacca cgtcaccagt actataacca
agagtggaca ttgtgggatc gctttgaggt acaagggctg
cagcctaatg gtgaggagat gaccctcaaa cagttcctcg
actattttaa gacagagcac aaattagaga tcaccatgct
gtcccagggc gtgtccatgc tctattcctt cttcatgcca
gctgccaagc tcaaggaacg gttggatcag ccgatgacag
agattgtgag ccgtgtgtcg aagcgaaagc tgggccgcca
cgtgcgggcg ctggtgcttg agctgtgctg taacgacgag
agcggcgagg atgtcgaggt tccctatgtc cgatacacca
tccgctgacc ccgtctgctc ctctaggctg gccccttgtc
cacccctctc cacacccctt ccagcccagg gttcccattt
ggcttctggc agtggcccaa ctagccaagt ctggtgttcc
ctcatcatcc ccctacctga acccctcttg ccactgcctt
ctaccttgtt tgaaacctga atcctaataa agaattaata
actcccaaaa aaaaaaaaaa aaaa
13. (Human E2 (Ubc13)-Accession NM_003348)

SEQ ID NO: 13

cgcgcgcgca gtcgcgcgcg ggtcgtgccg taccaccgtc
gcgggcaggc tcggccacga gcgccagagc cccgcgcctc
ccctcgcggc ctgtcccaag tccctgcccc gcaacagagc
gtcacttccg ccatccccgg cagcggttgg ggcggggcgc
acgggggagg gggccaggtc ggagggaagc ccgcccgtgc
ccgagcccgc gcccgagcag ggactacatt tcccgagggg
cctcggcggc ggctgcggcg acgggcgcgg caacgtcccc
cggaagtgga gcccgggact tccactcgtg cgtgaggcga
gaggagccgg agacgagacc agaggccgaa ctcgggttct
gacaagatgg ccgggctgcc ccgcaggatc atcaaggaaa
cccagcgttt gctggcagaa ccagttcctg gcatcaaagc
cgaaccagat gagagcaacg cccgttattt tcatgtggtc
attgctggcc ctcaggattc cccctttgag ggagggactt
ttaaacttga actattcctt ccagaagaat acccaatggc
agcccctaaa gtacgtttca tgaccaaaat ttatcatcct
aatgtagaca agttgggaag aatatgttta gatattttga
aagataagtg gtccccagca ctgcagatcc gcacagttct
gctatcgatc caggccttgt taagtgctcc caatccagat
gatccattag caaatgatgt agcggagcag tggaagacca
acgaagccca agccatagaa acagctagag catggactag
gctatatgcc atgaataata tttaaattga tacgatcatc
aagtgtgcat cacttctcct gttctgccaa gacttcctcc
tctttgtttg catttaatgg acacagtctt agaaacatta
cagaataaaa aagcccagac atcttcagtc ctttggtgat
taaatgcaca ttagcaaatc tatgtcttgt cctgattcac
tgtcataaag catgagcaga ggctagaagt atcatctgga
ttgttgtgaa acgtttaaaa gcagtggccc ttattcattt
cccccatcct ggtttaagta taaagcactg tgaatgaagg
tagttgtcag gttagctgca ggggtgtggg tgtttttatt
ttattttatt ttattttatt tttgaggggg gaggtagttt
aattttatgg gctcctttcc cccttttttg gtgatctaat
tgcattggtt aaaagcagct aaccaggtct ttagaatatg
ctctagccaa gtctaacttt atttagacgc tgtagatgga
caagcttgat tgttggaacc aaaatgggaa cattaaacaa
acatcacagc cctcactaat aacattgctg tcaagtgtag
attcccccct tcaaaaaaag cttgtgacca ttttgtatgg
cttgtctgga aacttctgta aatcttatgt tttagtaaaa
tattttttgt tattctactt tgcctttgta cagtttattt
tactgtgttt atttcatttt cccaatttga caatcgtatt
ttaaaattga aactgatgga acattctttc ttggtcttca
ccatctgaca aattgaatgg caagaggtgg attttgccag
tttcttttca ctgatgcaga tttgtgttaa gatagtactg
aatggagtat ttataaactg gccctgagca tgcataaagc
atcagtatct gacctttttt taaccttcta ggaatttgaa
ataaatgtgt ttgtgttgtc tgattagatg atcattggtg
tcttgccaca atgtttaaaa attactgtac aggaaagtca
cagcaaagat agcagttgtg actgacatgt aggactttca
cagttgtgcc acatttttgc ctaaaatttg ggttatgaca
tttttcttgg ttcttatctg aaaatttcat ctgtaacctt
tcatgtgtgt taagaaacac tgatctgatc atttgggatt
tgctgaggca tttgtgagtc ttccttataa acctgatgag
cagatctcaa ctatctagct tgtgtgtcat cagaaaggtt
tatccctttg agagtatcaa gtcctcagtt aatgattctt
gctttcatcc ctccagtatt tgctgtggga gctcgtttta
ttctttaatt tggaattcag taatttttct tctttattga
cgaattcctc ccctcacaaa actgttcttt cccacctctc
tccatatcta attcctgatt cttgttattt ttaagtcata
aatgtagcca gtcataaata cataaatgtt aaccttcggg
ttgcaacctt gtctcttgca gtttaaggta atggatattg
tagcccattt gaattttctt cactcttatt ctcgtaattc
tggagtttct tcagattgtg gtgtatttta ttgtgctcct
atgtaagatg aagaattaac tattaaaatt acattttcaa
catacaaaag cttttgatga ctggtaactg gtatccttcc
aaataaatgc attgcttggt aaaaaaaaaa aaaaaaaa
14. (Human ubiquitin E2 variant 2 (Mms2)-nucleic
acid-Accession NM_003350)

SEQ ID NO: 14

cgcgtcgggc tgcaggagaa gatggcggtc tccacaggag
ttaaagttcc tcgtaatttt cgcttgttgg aagaacttga
agaaggacaa aaaggagtag gcgacggtac agttagctgg
ggccttgaag atgatgaaga tatgacactt acaaggtgga
caggcatgat tattgggcca ccaaggacaa attatgaaaa
cagaatatat agcctgaaag tagaatgtgg acctaaatac
ccagaagctc ctccgtcagt tagatttgta acaaaaatta
atatgaacgg aataaataat tccagtggga tggtggatgc
ccggagcata ccagtgttag caaaatggca aaattcatat
agcattaaag ttgtacttca agagctaaga cgtctaatga
tgtccaaaga aaatatgaag cttccacagc caccagaagg
acaaacatac aacaattaat tttagtggat ctcaaacttg
tcttaaatca acaaccttct actcatgtta atgtcttgat
taaatatcac aatgcaaaat acacattaag taaaagaatt
ccagctggta aacatgacct ggacatttgt aagaatatat
ttaatatatg tacacccatt atgttttcag gtaacaggag
gaaaaatgca gcacaatttt ttttctcttg aaaggcactg
tcatttaaac ataaacctgg agtactcgaa atagaattca
ggtttacaag atgaaagcgt gtggagaagt gtcagatggc
agtggaagca tgtgtgtttc taaaaagtaa aaatctcaag
aaaacagaaa tggcatgctt tacccatctt acttagtgaa
agagagctgc agttgaaatt gtttaaaaag tagcaggtac
aatgaatatt gtcacagatg tgttaatttt tgaagcaatg
tgggtgctga ctactagtag tatcaaaaat atgttcagga
ttgttttgat acctgtattt ataataaaaa atgttggggg
gagttgatga attcctgtta aaagctgttc ttgtgtgtta
catgtaacag acatggtaaa tatttgttta cagtctttgt
ttaacaaacc atgcatttaa gtttaagtga agtcaacaaa
aaggaaatag gtgtatggat atgtgatttt gagattaaag
ttagtcttaa aatgtaaata aaatgtgaaa cgtgtcctca
gagactgtgc catttctatt atgttgatgt atatgtacag
taccttgcca gggaagcaaa aattggaatt attgtagctt
ttcatgtata cacactttta tttaccctat tttgtgtact
tcttgtgaat tataatttgc agactatttc agaaaagaaa
ttatctagtt taatttcttc tttggacaag gagtcctagg
tattatattt tgagtttgat ttcaccagaa ataataatat
taaaaagatc tttgcattct ggcagttctt ttaggattat
aggttgcaaa ttatccaaat atatatccca ttttttaaag
cataaaaaaa aaaaa
15. (Human ubiquitin E2 variant 2 (Mms2)-Accession
NM_003341.1)

SEQ ID NO: 15

mavstgvkvp rnfrlleele egqkgvgdgt vswgledded
mtltrwtgmi igpprtnyen riyslkvecg pkypeappsv
rfvtkinmng innssgmvda rsipvlakwq nsysikvvlq
elrrlmmske nmklpqppeg qtynn

What is claimed is:

1. A method of identifying a ubiquitination modulator comprising
(a) combining, under conditions that favor ubiquitination activity ubiquitin, a candidate modulator, ubiquitin activating enzyme (E1) and ubiquitin conjugating enzyme (E2), thereby producing a reaction mixture, and
(b) measuring the amount of polyubiquitin, whereby a difference in polyubiquitin as compared with a reaction performed in the absence of the candidate modulator indicates that the candidate is a ubiquitination modulator.

2. The method of claim 1, wherein the reaction mixture further comprises adenosine tri-phosphate (ATP).

3. The method of claim 1, wherein the reaction mixture substantially lacks ubiquitin ligase (E3).

4. A method of identifying a ubiquitination modulator comprising:
(a) combining, under conditions that favor ubiquitination activity:
(i) tag1-ubiquitin,
(ii) tag2-ubiquitin,
(iii) a candidate modulator,
(iv) ubiquitin activating enzyme (E1), and
(v) ubiquitin conjugating enzyme (E2), thereby producing a reaction mixture; and
(b) measuring the amount of tag1-ubiquitin bound to said tag2-ubiquitin in said reaction mixture, whereby a difference in bound ubiquitin as compared with a reaction performed in the absence of the candidate modulator indicates that the candidate is a ubiquitination modulator.

5. The method of claim 4, wherein the reaction mixture further comprises adenosine tri-phosphate (ATP).

6. The method of claim 4, wherein the reaction mixture substantially lacks ubiquitin ligase (E3).

7. The method of claim 4, wherein ubiquitin conjugating enzyme (E2) comprises Ubiquitin conjugating enzyme 13 (Ubc13).

8. The method of claim 4, wherein ubiquitin conjugating enzyme (E2) comprises ubiquitin E2 variant 1a (Uev1a).

9. The method of claim 4, wherein ubiquitin conjugating enzyme (E2) comprises Ubiquitin conjugating enzyme 13 (Ubc13) and ubiquitin E2 variant 1a (Uev1a).

10. The method of claim 4, wherein tag1 and tag2 are fluorescent labels constituting a fluorescence resonance energy transfer (FRET) pair.

11. The method of claim 4, wherein ubiquitin conjugating enzyme (E2) comprises Ubiquitin conjugating enzyme 13 (Ubc13) and ubiquitin E2 variant 2 (Mms2).
